SEQUENCE LISTING (30 sequences)

SEQ ID NO: 1 - DNA, 933 bp, Artificial Sequence
  rd29 promoter comprising one deletion
ccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctt

SEQ ID NO: 2 - DNA, 934 bp, Arabidopsis thaliana
ccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctt

SEQ ID NO: 3 - DNA, 30 bp, Artificial Sequence
  primer naste 8
gcccgggccatagatgcaattcaatcaaac

SEQ ID NO: 4 - DNA, 41 bp, Artificial Sequence
  primer naste 9
gcgctagcctcgagttaattaagatttttttctttccaata

SEQ ID NO: 5 - DNA, 3000 bp, Artificial Sequence
  vector pGEM-T
gggcgaattgggcccgacgtcgcatgctcccggccgccatggccgcgggatatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 6 - DNA, 3976 bp, Artificial Sequence
  vector pNVS10
atcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgattgcccgggccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattgaaagaaaaaaatcttaattaactcgaggctagcgc

SEQ ID NO: 7 - DNA, 4205 bp, Artificial Sequence
  vector pNVS11
ggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgattgcccgggccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattgaaagaaaaaaatcttaattaactcgaggctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgca

SEQ ID NO: 8 - DNA, 31 bp, Artificial Sequence
  Primer pNVS11FW
cctcatgaccatagatgcaattcaatcaaac

SEQ ID NO: 9 - DNA, 43 bp, Artificial Sequence
  Primer pNVS11RV
ccgctagcgcatccatggtccaaagatttttttctttcaatag

SEQ ID NO: 10 - DNA, 4180 bp, Artificial Sequence
  vector pNVS122
ggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatgaccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattgaaagaaaaaaatctttggaccatggatgcgctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgca

SEQ ID NO: 11 - DNA, 48 bp, Artificial Sequence
  Primer naste 79
ccgctagcgcatccatggtccaaagatttttttctttccaatagaagt

SEQ ID NO: 12 - DNA, 4182 bp, Artificial Sequence
  vector pNVS123
ggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatgaccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaccatggatgcgctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgca

SEQ ID NO: 13 - DNA, 55 bp, Artificial Sequence
  Primer naste75
catgcccgggcgcgcctgtacagcggccgcgaattcgttaactctagagcgatcg

SEQ ID NO: 14 - DNA, 56 bp, Artificial Sequence
  Primer naste 80
ccgggcgatcgctctagagttaacgaattcgcggccgctgtacaggcgcgcccggg

SEQ ID NO: 15 - DNA, 4244 bp, Artificial Sequence
  vector pNVS124
ggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatgcccgggcgcgcctgtacagcggccgcgaattcgttaactctagagcgatcgcggccgccatgaccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaccatggatgcgctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgca

SEQ ID NO: 16 - DNA, 15171 bp, Artificial Sequence
  vector pTIBE10
attatacatagttgaagcttctgcaggtcgaggagaaatatgagtcgaggcatggatacactaagttcccctgaagtgagcatgatctttgatgctgagatgattcccagagcaagatagtttgtgctgcaagtgacacaattgtaatgaaaccaccactcaacgaatttacttgtggctttgacatgtcgtgtgctctgtttgtatttgtgagtgccggttggtaattatttttgttaatgtgattttaaaacctcttatgtaaatagttactttatctattgaagtgtgttcttgtggtctatagtttctcaaagggaaattaaaatgttgacatcccatttacaattgataacttggtatacacaaactttgtaaatttggtgatatttatggtcgaaagaaggcaatacccattgtatgttccaatatcaatatcaatacgataacttgataatactaacatatgattgtcattgtttttccagtatcaatatacattaagctactacaaaattagtataaatcactatattataaatctttttcggttgtaacttgtaattcgtgggtttttaaaataaaagcatgtgaaaattttcaaataatgtgatggcgcaattttattttccgagttccaaaatattgccgcttcattaccctaatttgtggcgccacatgtaaaacaaaagacgattcttagtggctatcactgccatcacgcggatcactaatatgaaccgtcgattaaaacagatcgacggtttatacatcattttattgtacacacggatcgatatctcagccgttagatttaatatgcgatctgattgctcaaaaaatagactctccgtctttgcctataaaaacaatttcacatctttctcacccaaatctactcttaaccgttcttcttcttctacagacatcaatttctctcgactctagaggatccaatcttatcgatttcgaacccctcaggcgaagaacaggtatgatttgtttgtaattagatcaggggtttaggtctttccattactttttaatgttttttctgttactgtctccgcgatctgattttacgacaatagagtttcgggttttgtcccattccagtttgaaaataaaggtccgtcttttaagtttgctggatcgataaacctgtgaagattgagtctagtcgatttattggatgatccattcttcatcgtttttttcttgcttcgaagttctgtataaccagatttgtctgtgtgcgattgtcattacctagccgtgtatcgagaactagggttttcgagtcaattttgccccttttggttatatctggttcgataacgattcatctggattagggttttaagtggtgacgtttagtattccaatttcttcaaaatttagttatggataatgaaaatccccaattgactgttcaatttcttgttaaatgcgcagatccccatggcttcgatctcctcctcagtcgcgaccgttagccggaccgcccctgctcaggccaacatggtggctccgttcaccggccttaagtccaacgccgccttccccaccaccaagaaggctaacgacttctccacccttcccagcaacggtggaagagttcaatgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatggccggcgccgaggagatcgtgctgcagcccatcaaggagatctccggcaccgtcaagctgccggggtccaagtcgctttccaaccggatcctcctactcgccgccctgtccgaggggacaacagtggttgataacctgctgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtccttgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccgtacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggcgatggccttctcccttgccgcctgtgccgaggtccccgtcaccatccgggaccctgggtgcacccggaagaccttccccgactacttcgatgtgctgagcactttcgtcaagaattaagctctagaactagtggatcccccgatccgcgtttgtgttttctgggtttctcacttaagcgtctgcgttttacttttgtattgggtttggcgtttagtagtttgcggtagcgttcttgttatgtgtaattacgctttttcttcttgcttcagcagtttcggttgaaatataaatcgaatcaagtttcactttatcagcgttgttttaaattttggcattaaattggtgaaaattgcttcaattttgtatctaaatagaagagacaacatgaaattcgacttttgacctcaaatcttcgaacatttatttcctgatttcacgatggatgaggataacgaaagggcggttcctatgtccgggaaagttcccgtagaagacaatgagcaaagctactgaaacgcggacacgacgtcgcattggtacggatatgagttaaaccgactcaattcctttattaagacataaaccgattttggttaaagtgtaacagtgagctgatataaaaccgaaacaaaccggtacaagtttgattgagcaacttgatgacaaacttcagaattttggttattgaatgaaaatcatagtctaatcgtaaaaaatgtacagaagaaaagctagagcagaacaaagattctatattctggttccaatttatcatcgctttaacgtccctcagatttgatcgggctgcaggaattaaacgcccgtagcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacaaattacaacggtatatatcctgccagtactcggccgtcgacctgcaggttaactcgagctcgaattcacgcgtgggcccgggcgcgcccaactttgtatagaaaagttggccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaacaagtttgtacaaaaaagcaggctatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgacccagctttcttgtacaaagtggggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatccaacttt

SEQ ID NO: 17 - DNA, 15200 bp, Artificial Sequence
  vector pTIBE28
cgcgcctgtacagcggccgcgaattcgttaactctagagcgatcgcggccgccatgaccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcggggactagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgcaggtcgacggccgagtactggcaggatatataccgttgtaatttgtcgcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgccgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatccgaatctggcaattccggttcgcttgctgtccataaaaccgcccagtctagctatcgccatgtaagcccactgcaagctacctgctttctctttgcgcttgcgttttccggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgcccggacggccgaggtcttccgatctcctgaagccagggcagatccgtgcacagcacttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggctccatggcgatcgctacgggcgtttaattcctgcagcccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcgggggatccactagttctagagcttaattcttgacgaaagtgctcagcacatcgaagtagtcggggaaggtcttccgggtgcacccagggtcccggatggtgacggggacctcggcacaggcggcaagggagaaggccatcgccatcctgtggtcgtcgtacgtgtcgatcgccgtcacgttcagcttctccggcggcgtgatgatgcagtagtccggcccttcctcaacagatgctcccagcttggttagctccgtccggatcgcaaccatcctctcggtctcctttactctccaggaagccacgtctctgatggctgtcgggccatcggcaaagagggcaaccacagcaagagtcatggcgacatcaggcatcttgttcatgttgacatcaatcgccttgaggtgtttcctcccaaatggctcccgcggtgggccagtaacagttacgctagtctcggtccatgtaaccttcgctcccatcatctccagtacctcagcaaacttcacatcaccctgcaaactggtggtgccacaaccttccacagtcacagtccctccagtaattgcagcaccagccaagaaatagcttgcgcttgaggcatcaccttcaacataggcatttttaggggacttgtatttttgacctcccttaatgtagaatctgtcccagctatcagaatgctctgctttcacaccaaaacgctccatcaatctcaatgtcatttcgacgtacggaatggagattaatttatcaatgatttcaatctccacatccccaagagccaaaggagcagccatcagcaaggcactcaagtactgactgctgatggagccagacagcttgaccttgccaccaggtagccctccgattccattgacacgaacaggtgggcagtcagtgccaaggaaacaatcaacatctgcaccaagctgcttcaatccgacaaccaagtcgccaatgggtctctccctcattcttggtactccatcaagcacgtaagttgcatttccaccagcagcagtaacagctgctgtcaaggaccgcattgcgattccagcattccccaagaagagctgcacttcctctttagcatcctcaactgggaactttccaccacagccaacaactacagctcttttggcagctttgtccgcttcgacagagagaccaagagtcctcaaggccccgagcatgtagtggacatcctcactgttcagcaggttatcaaccactgttgtcccctcggacagggcggcgagtaggaggatccggttggaaagcgacttggaccccggcagcttgacggtgccggagatctccttgatgggctgcagcacgatctcctcggcgccggccatgcaccggatccttccgccgttgctgacgttgccgaggcttctggaggagcggcgggcgacggggaggctggcggtggacttgagcccctggaacggagcgacggcggtggccgacgaggccatcatcacggtgggcgccatagacagcggcggcaggtacgacagcgtctcgaacttcttgttgccgtaggccggccacacctgcatacattgaactcttccaccgttgctgggaagggtggagaagtcgttagccttcttggtggtggggaaggcggcgttggacttaaggccggtgaacggagccaccatgttggcctgagcaggggcggtccggctaacggtcgcgactgaggaggagatcgaagccatggggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgaggggttcgaaatcgataagcttggatcctctagagtcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgccacaaattagggtaatgaagcggcaatattttggaactcggaaaataaaattgcgccatcacattatttgaaaattttcacatgcttttattttaaaaacccacgaattacaagttacaaccgaaaaagatttataatatagtgatttatactaattttgtagtagcttaatgtatattgatactggaaaaacaatgacaatcatatgttagtattatcaagttatcgtattgatattgatattggaacatacaatgggtattgccttctttcgaccataaatatcaccaaatttacaaagtttgtgtataccaagttatcaattgtaaatgggatgtcaacattttaatttccctttgagaaactatagaccacaagaacacacttcaatagataaagtaactatttacataagaggttttaaaatcacattaacaaaaataattaccaaccggcactcacaaatacaaacagagcacacgacatgtcaaagccacaagtaaattcgttgagtggtggtttcattacaattgtgtcacttgcagcacaaactatcttgctctgggaatcatctcagcatcaaagatcatgctcacttcaggggaacttagtgtatccatgcctcgactcatatttctcctcgacctgcagaagcttgg

SEQ ID NO: 18 - DNA, 2001 bp, Artificial Sequence
  nucleic acid sequence encoding GUS
  Intron (587)..(776)
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 19 - DNA, 224 bp, Artificial Sequence
  3′ CaMV 35S terminator
ggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 20 - DNA, 1336 bp, Artificial Sequence
  2mepsps selectable marker
ccggcgccgaggagatcgtgctgcagcccatcaaggagatctccggcaccgtcaagctgccggggtccaagtcgctttccaaccggatcctcctactcgccgccctgtccgaggggacaacagtggttgataacctgctgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtccttgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccgtacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggcgatggccttctcccttgccgcctgtgccgaggtccccgtcaccatccgggaccctgggtgcacccggaagaccttccccgactacttcgatgtgctgagcactttcgtcaagaattaagc

SEQ ID NO: 21 - DNA, 2301 bp, Artificial Sequence
  NheI-NcoI cut GUS gene with intron and the caMV 3′ 35S terminator
  gene (1)..(2001): GUS gene with intron
  terminator (2078)..(2301)
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcggggactagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 22 - DNA, 651 bp, Artificial Sequence
  nucleic acid sequence encoding the PNC1 protein
atgaaaacactcatcgttgtggatatgcagaacgatttcataagtcctcttggatctctaactgtaccaaagggcgaagaactcataaatcccatttctgatctcatgcaagatgctgatagagattggcataggatagttgttactagagattggcatccaagcagacatatttcctttgccaagaatcataaggacaaggaaccctattccacttatacctatcatagtccaagacctggagatgattcaactcaagaaggtatactatggcctgttcattgcgttaagaatacatggggttcgcaacttgttgatcaaattatggatcaggtggtaaccaagcacattaagattgtggataaggggtttcttaccgatagagagtactattctgctttccatgacatttggaacttccacaagacagatatgaataagtacctggagaagcatcatactgacgaagtttacattgttggtgttgcattggagtattgtgtaaaggctactgcaatatcagcagctgaacttggttacaaaacaactgtgctgcttgattacacaagacctatttcagacgatccagaagttatcaacaaggttaaggaagagcttaaggctcacaatatcaacgttgtagacaagtga

SEQ ID NO: 23 - DNA, 1206 bp, Artificial Sequence
  nucleic acid sequence encoding the NMA1 protein
atggatccaacaagagcaccagatttcaaaccacctagtgctgatgaagaactaattccacctccagatcctgaatcaaagattcctaagtccatcccaatcattccttatgtgcttgctgatgccaattcatctatagacgcacccttcaatatcaagcgaaagaagaagcatcctaagcatcatcaccatcatcatcacagtagaaaggaaggaaacgacaagaagcatcaacatattccgctcaaccaagatgatttccaacctctttctgctgaagtatcttctgaagatgatgacgctgatttccgttcaaaggaaaggtatggttctgattcgaccactgaatctgaaactagaggtgtacagaagtaccaaattgctgatctcgaagaagttccacatggtatagttagacaagccagaactcttgaagattacgagttccctagccataggctttctaagaaactgcttgatcccaataagctcccactagttattgttgcatgcggttctttctctcccattacctatctgcatcttagaatgttcgagatggctcttgatgccatttcagaacagacaagattcgaggtaataggaggctactatagtcctgttagcgataactatcagaagcaaggattggcaccatcttatcatagagtgagaatgtgtgaactggcttgtgaaagaacttcttcatggcttatggttgatgcatgggaatctcttcaaccttcttacacaagaactgccaaggttcttgatcatttcaaccacgagataaacattaagagaggaggtgttgcaactgttactggtgaaaagattggcgtcaagattatgttacttgctggaggtgaccttattgaaagtatgggagaacctaatgtttgggctgatgctgatttgcatcacattcttggcaactatggttgccttatagttgaaagaaccggatctgatgttcgttcttttctcctttcccacgatataatgtacgagcacagacgtaacatcttgataatcaagcagctcatctacaatgacatctcttcaacaaaggttcgacttttcatacgaagagccatgagtgtacaatatcttctgcccaactccgttattagatacattcaggagcacagactttacgttgatcaaacagaacctgtgaagcaagttcttggtaataaggagtaa

SEQ ID NO: 24 - DNA, 134 bp, Artificial
  nucleic acid encoding a microRNA against PARP1
tctagggtaagggaggtagagtaagaacacttgctcacagggtactttcttgcatgcttgagcctttcatgcttgaagctctgcgagcaagtcttctatactctatccacttttctctctctctctcactcact

SEQ ID NO: 25 - DNA, 1560 bp, Artificial
  nucleic acid encoding a hairpin RNA against PARP2
aaattatacagttgatgttgttcaaatattcaaggtgacaagagacggtgaaagtgaacgctttaaaaagttttctggaacaaaaaatagaatgctgttgtggcatggttctcggcttactaactggactggcattctgtcccaaggtttgcgcattgctccacctgaagcgcctgccacgggttatatgtttgggaagggggtttactttgctgatatgttctccaaaagtgcaaattattgctatactaattctgccttcacaacaggggtgttgcttctatgtgaggttgccctgggtgacatggctgagcttctacaagctaggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgattagcttgtagaagctcagccatgtcacccagggcaacctcacatagaagcaacacccctgttgtgaaggcagaattagtatagcaataatttgcacttttggagaacatatcagcaaagtaaacccccttcccaaacatataacccgtggcaggcgcttcaggtggagcaatgcgcaaaccttgggacagaatgccagtccagttagtaagccgagaaccatgccacaacagcattctattttttgttccagaaaactttttaaagcgttcactttcaccgtctcttgtcaccttgaatatttgaacaacatcaactgtataatttgaatgtgtctgagcatgagtattctgaatatactttacaatcaaagcgaactcctcagtatcattgtcaagaggaaacagttcacagtgaagctgctggtat

SEQ ID NO: 26 - DNA, 1264 bp, Artificial
  nucleic acid encoding a hairpinRNA directed against farnesytransferase alpha FTA)
cggattgctaattctaactctaagaattatcagttatggcaccatcggcggtgggttgtcgagagattgggagctaatgctagagccaaggagcttaatctcattaagaagatactatcaattgatgccaaaaactatcatgcctggtcacataggcagtgggtgcttcaggcattaggaggctgggaagatgaacttgattattgtcagcaacttcttgaaggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttggtaccttcaagaagttgctgacaataatcaagttcatcttcccagcctcctaatgcctgaagcacccactgcctatgtgaccaggcatgatagtttttggcatcaattgatagtatcttcttaatgagattaagctccttggctctagcattagctcccaatctctcgacaacccaccgccgatggtgccataactgataattcttagagttagaattagcaatccg

SEQ ID NO: 27 - DNA, 1160 bp, Artificial
  nucleic acid encoding a hairpinRNA directed against farnesytransferase beta (FTB)
gtagatcctgaattggaaaataatactattgattttcttagtcgttgtcagggtcctaatggtggttatgctggtggacctggacagatgcctcaccttgcaacaacatatgctgcagtcaattcacttgttactctgggtggtgacaaagcattgtcatcaattaatagggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttggtaccctattaattgatgacaatgctttgtcaccacccagagtaacaagtgaattgactgcagcatatgttgttgcaaggtgaggcatctgtccaggtccaccagcataaccaccattaggaccctgacaacgactaagaaaatcaatagtattattttccaattcaggatctac

SEQ ID NO: 28 - DNA, 2460 bp, Artificial
  nucleic acid sequence encoding the Los5 protein
atggaagcatttcttaaggaattcggagattattatggatacccagatggtcccaagaacattcaagagatccgcgacaccgaattcaagagattagataaaggtgttgtatacttggatcatgctggttctactttgtattctgagttgcagatggaatatatttttaaggacttcacaagcaatgtttttggaaatccacatagtcaaagtgatatcagttcggccaccagtgaccttatagcggatgctcgacatcaggtgcttgaatactttaatgcatctcctgaagattacagttgcttattcacctccggagccacagcagcgctgaagcttgtcggagagacttttccgtggacccaagacagtaattttttgtataccatggagaatcacaacagtgtacttggtattagggaatatgcattagctcaaggtgcttcagcatgtgcagtggatattgaagaggcagctaaccaaccaggccagcttacaaattcaggaccatctatcaaggtaaagcatcgtgctgtgcagatgagaaacacttctaaactccaaaaggaagagtcaagaggaaatgcctataatctatttgctttcccctcggagtgcaatttttctggcctgaggtttaatctagatctggtgaagttgatgaaagaaaatactgagaccgtgctacaaggctccccctttagcaagagcaagcggtggatggtcttgattgatgctgcaaagggttgtgctacactaccacctgatttatcggagtatcctgcagattttgttgttctgtcattctacaagttatttggttatcctactgggcttggcgctctccttgtacggaatgatgcagccaaattgctcaaaaagacttattttagtggaggcactgttgctgcttcaattgctgacatcgactttgtaaaaagaagggaaagggtggaggagttttttgaggatggttctgcgtcattcctgagcatagcagccatccgtcatggcttcaaattactcaagtcgcttacaccttctgcaatttggatgcacacaacgtcactttccatatatgtgaaaaagaagcttcaggctttacgacatggaaacggggctgctgtatgtgttctgtatggcagtgaaaatctggagttatcttcacataaatcaggcccaacggttacattcaacttgaaaagacctgatggctcttggtttggctacttggaggtggagaagcttgcttctttatctggaattcagttacggacaggatgtttttgcaatcctggcgcatgtgcaaagtatctcgagttatcccattctgagctacggtctaatgtagaggctgggcatatttgctgggatgacaatgatgtgataaatggaaaaccaacaggggctgttagggtttcgtttggttatatgtcaacctttgaagatgccaagaaatttattgatttcatcataagttcatttgcttcacctccaaagaagactgggaatggaaccgtcgtcagtggaaggtttcctcaacttcctagtgaagaccttgaaagtaaagaatcttttccaagccactaccttaagtcaattactgtatacccgatcaagtcatgtgctggattttctgtgatacgttggccactttgcagaacaggcctgctgcatgatcgagaatggatggttcagggtctgaccggtgaaattcttacccaaaagaaggtgcctgagatgtctcttataaaaacctttatcgaccttgaggaaggactactgtctgtagaatcttctcgctgcgaagacaagttgcacatcagaatcaagtctgattcatataacccgaggaacgatgagtttgattcacatgccaacatacttgaaaaccgtaatgaggaaactagaatcaatcgttggttcaccaatgccattggtcgacaatgcaagttgctacggtattctagctctacttccaaagactgcttgaacagaaacaagagtcctggtttgtgcagagatttggaaagcaatatcaactttgctaatgaagctcagttcttgttaatctccgaggagagtgttgctgacctaaacagaagattagaagcaaaagacgaggattacaaacgggctcatgaaaaactcaatccacataggttcagaccaaatctggttatatctggaggtgaaccatacggggaagataaatggaaaactgtcaagataggagacaatcatttcacatcattgggcggttgtaaccggtgccagatgataaacataagtaatgaagctggactagtgaagaaatccaatgagcccttaacaactttagcttcatataggagagtaaagggaaagatcttgtttggaacgcttttgagatacgagattgatgagaaaagacaatgttggattggagttggggaagaagttaatccagatattgaataa

SEQ ID NO: 29 - DNA, 10 bp, Artificial
  3′ end of rd29a promoter region
tctttggaaa

SEQ ID NO: 30 - DNA, 10 bp, Artificial
  modified 3′ end of rd29a promoter region
tcttaattaa

(19) United States
(12) Patent Application Publication
(10) Pub. No.: US 2017/0260539 A1
(43) Pub. Date: Sep. 14, 2017

(54) USE OF THE RD29 PROMOTER OR FRAGMENTS THEREOF FOR STRESS-INDUCIBLE EXPRESSION OF TRANSGENES IN COTTON

(71) Applicant: Bayer CropScience NV, Diegem (BE)

(72) Inventors: Stephane PIEN, Bergisch Gladbach (DE); Bart DEN BOER, Merelbeke (BE)

(21) Appl. No.: 15/599,866

(22) Filed: May 19, 2017

Related U.S. Application Data
(63) Continuation of application No. 13/978,582, filed on Jul. 15, 2013, now abandoned.
(60) Provisional application No. 61/435,495, filed on Jan. 24, 2011.

(30) Foreign Application Priority Data
Jan. 24, 2011 (EP) 11075010.6
Oct. 28, 2011 (EP) 11187147.1

(51) Int. Cl. C12N 15/82

(52) U.S. Cl. CPC C12N 15/8273; C12N 15/8218; C12N 15/8237; C12N 15/8271; C12N 15/8238

(57) ABSTRACT

In one aspect, the present application discloses a chimeric gene comprising (a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; (b) a second nucleic acid sequence encoding an expression product of interest, which is involved in the response of a cotton plant to stress; and optionally (c) a transcription termination and polyadenylation sequence. In another aspect, the application discloses a cotton plant cell comprising (a) a chimeric gene comprising a first nucleic acid sequence comprising at least 400 consecutive nucleotides of SEQ ID NO: 1 or SEQ ID NO: 2 or a nucleic acid sequence having at least 80 sequence identity thereto any of which confers stress inducibility on said chimeric gene; (b) a second nucleic acid sequence encoding an expression product of interest; and optionally (c) a transcription termination and polyadenylation sequence. In addition, the present application discloses a cotton plant, a method of expressing a transgene in cotton under stress conditions, a method of producing a cotton plant, a method of detecting the expression of a transgene under stress conditions and a method for modulating the resistance of a cotton plant to stress as characterized in the claims.

### CROSS-REFERENCE TO RELATED APPLICATIONS

This application is a continuation of U.S. application Ser. No. 13/978,582, filed Jul. 8, 2013, which is a §371 U.S. National Stage of International Application No. PCT/EP2012/051036, filed Jan. 24, 2012, which claims the benefit of European Patent Application Serial No. 11075010.6, filed Jan. 24, 2011, European Patent Application Serial No. 11187147.1, filed Oct. 28, 2011 and U.S. Patent Application Ser. No. 61/435,495, filed Jan. 24, 2011, the contents of which are herein incorporated by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named “bcs11_2002_WOST25.txt”, created on Nov. 14, 2013, and having a size of 95,000 bytes and is filed concurrently with the specification. The sequence listing contained in this ASCII formatted document is part of the specification and is herein incorporated by reference in its entirety.

### DETAILED DESCRIPTION

In one aspect, the present application discloses a chimeric gene comprising (a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; (b) a second nucleic acid sequence encoding an expression product of interest, which is involved in the response of a cotton plant to stress; and optionally (c) a transcription termination and polyadenylation sequence. In another aspect, the application discloses a cotton plant cell comprising (a) a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; (b) a second nucleic acid sequence encoding an expression product of interest; and optionally (c) a transcription termination and polyadenylation sequence. In addition, the present application discloses a cotton plant, a method of expressing a transgene in cotton under stress conditions, a method of producing a cotton plant, a method of detecting the expression of a transgene under stress conditions and a method for modulating the resistance of a cotton plant to stress as characterized in the claims.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In recent years the phenomenon of global warming and its effect on crop plant production has become a crucial issue. Solving this problem at the plant science level is almost exclusively a question of coping with plant stress. International agricultural and environmental research institutions now re-discover plant stress as a major component of the effect of global warming on local and global food production. Research to meet these challenges involves learning in widely diverging disciplines such as atmospheric sciences, soil science, plant physiology, biochemistry, genetics, plant breeding, molecular biology and agricultural engineering.

Abiotic plant environmental stress constitutes a major limitation to crop production. The major plant environmental stresses of contemporary economical importance worldwide are water stress including drought and flooding, cold (chilling and freezing), heat, salinity, water logging, soil mineral deficiency, soil mineral toxicity and oxidative stress. These factors are not isolated but also interrelated and influencing each other.

Abscisic acid (ABA) is a phytohormone which functions in many plant developmental processes, including bud dormancy. Furthermore, ABA mediates stress responses in plants in reaction to water stress, high-salt stress, cold stress (Mansfield 1987, Yamaguchi-Shinozaki 1993, Yamaguchi-Shinozaki 1994) and plant pathogens (Seo and Koshiba, 2002). ABA is a sesquiterpenoid (15-carbon) which is partially produced via the mevalonic pathway in chloroplasts and other plastids. It is sythesized partially in the chloroplasts and accordingly, biosynthesis primarily occurs in the leaves. The production of ABA is increased by stresses such as water loss and freezing temperatures. It is believed that biosynthesis occurs indirectly through the production of carotenoids.

Physiological responses known to be associated with abscisic acid include stimulation of the closure of stomata, inhibition of shoot growth, induction of storage protein synthesis in seeds and inhibition of the effect of gibberellins on stimulating de novo synthesis of a-amylase.

Basic ABA levels may differ considerably from plant to plant. For example, the basal concentration of ABA in non-stressed Arabidopsis leaves is 2 to 3 ng/g fresh weight (Lopez-Carbonell and Jauregui, 2005). Under water-stress conditions, the ABA concentration reaches 10 to 21 ng/g fresh weight. On the other hand, in non-stressed cotton, the concentration of ABA in leaves varies between 145 to 2490 ng/g fresh weight (Ackerson, 1982).

Genes involved in responses to abiotic stress as well as promoters mediating stress responses have been described in the art.

Already in 1994, Yamaguchi-Shinozaki and Shinozaki described and analyzed a promoter regulating the rd29A gene in Arabidopsis which is induced in response to dehydration, low temperature, high salt or treatment with exogenous abscisic acid.

A major challenge in agriculture practice and research today is how to cope with plant environmental stress in an economical and an environmentally sustainable approach. In view of the already existing regions exposed to abiotic stress conditions in the world and the ongoing climate change, the provision of transgenic plants conferring resistance on at least one kind of abiotic stress is still a major goal in order to achieve a satisfying nutritional situation also in regions exposed to such abiotic stress in the world.

Accordingly, in one example, the present application discloses a chimeric gene comprising (a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; (b) a second nucleic acid sequence encoding an expression product of interest, which is involved in the response of a cotton plant to stress; and optionally (c) a transcription termination and polyadenylation sequence.

Unless indicated otherwise, the embodiments described below for the chimeric gene disclosed herein are also applicable to respective embodiments of other aspects disclosed herein.

As used herein, the term “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc. However, in context with the present disclosure, the term “comprising” also includes “consisting of”.

A chimeric gene is an artificial gene constructed by operably linking fragments of unrelated genes or other nucleic acid sequences. In other words “chimeric gene” denotes a gene which is not normally found in a plant species or refers to any gene in which the promoter or one or more other regulatory regions of the gene are not associated in nature with a part or all of the transcribed nucleic acid, i. e. are heterologous with respect to the transcribed nucleic acid. More particularly, a chimeric gene is an artificial, i. e. non-naturally occurring, gene produced by an operable linkage of the first nucleic acid sequence comprising at least 400 consecutive nucleotides of SEQ ID NO: 1 or SEQ ID NO: 2 or a nucleic acid sequence having at least 80% sequence identity thereto any of which confer stress inducibility on said chimeric gene with a second nucleic acid sequence encoding an expression product of interest which is not naturally operably linked to said nucleic acid sequence. Such nucleic acid sequence naturally operably linked to said first nucleic acid sequence is the coding sequence of the rd29A gene.

The term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism). For example, the chimeric gene disclosed herein is a heterologous nucleic acid.

Nucleic acids can be DNA or RNA, single- or double-stranded. Nucleic acids can be synthesized chemically or produced by biological expression in vitro or even in vivo. Nucleic acids can be chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill. , USA), Glen Research (Sterling, Va., USA), ChemGenes (Ashland, Mass., USA), and Cruachem (Glasgow, UK).

In connection with the chimeric gene of the present disclosure, DNA includes cDNA and genomic DNA.

Said first nucleic acid sequence confers stress inducibility on the chimeric gene disclosed herein. Likewise, said first nucleic acid sequence confers inducibility of the expression of the second nucleic acid sequence encoding an expression product of interest described further below in response to abiotic stress conditions. In other words, expression of said chimeric gene is induced upon exposure of a plant comprising said chimeric gene to stress. In this regard, stress includes abiotic stresses such as water stress, drought stress, cold stress, high-salt stress and the application of ABA.

The length of the first nucleic acid sequence and its position within SEQ ID NO: 1 or SEQ ID NO: 2 is to be chosen such that it is sufficiently long and positioned such that expression of the chimeric gene comprising it is induced upon exposure to stress. Methods of evaluating whether a first nucleic acid sequence, which in the present application represents a promoter sequence, is capable of inducing expression of the chimeric gene it is comprised in or, in particular, the nucleic acid sequence operably linked thereto, upon exposure to stress are known to the skilled person. For example reporter gene studies may be performed in order to evaluate the inducing function of said first nucleic acid under stress conditions. This includes operably linking said first nucleic acid sequence to a reporter gene such as GUS (beta-glucuronidase) or GFP (green fluorescent protein), transforming the resulting nucleic acid construct or chimeric gene into a plant or plant cell, in this case a cotton plant, and evaluating induction of the expression of said reporter gene upon exposure of the plant or plant cell to stress such as water stress such as drought stress, cold stress, high-salt stress or exposure to ABA in comparison with a plant or plant cell not comprising said construct. Said first nucleic acid sequence conferring stress inducibility in some examples may accordingly comprise at least 400, at least 450, at least 500, at least 550, at least 600, at least 650, at least 700, at least 800 or at least 900 consecutive nucleotides of SEQ ID NO: 1 or SEQ ID NO: 2. In another example, said first nucleic acid sequence comprises the nucleotide sequence of SEQ ID NO: 1 or SEQ ID NO: 2. In yet another example, said first nucleic acid sequence consists of SEQ ID NO: 1 or SEQ ID NO: 2.

In one aspect, nucleic acid sequences for promoters capable of conferring stress inducibility on a chimeric gene, in particular nucleic acid sequences comprising a nucleotide sequence having at least 70%, at least 80%, at least 90%, at least 95% or at least 98% sequence identity to SEQ ID NO: 1 or SEQ ID NO: 2 are provided. Such nucleic acid sequences also include artificially derived nucleic acid sequences, such as those generated, for example, by using site-directed mutagenesis of SEQ ID NO: 1 or SEQ ID NO: 2. Generally, nucleotide sequence variants disclosed herein may have at least 70%, such as 72%, 74%, 76%, 78%, at least 80%, e.g., 81% to 84%, at least 85%, e.g., 86%, 87%, 88%, 89%, 90%, 91%, 92%, 93%, 94%, 95%, 96%, 97%, to 98% and 99% sequence identity to the nucleic acid sequence of SEQ ID NO: 1 or SEQ ID NO: 2. Sequence identity is calculated based on the shorter nucleotide sequence. Nucleic acid sequences disclosed herein may also include, but are not limited to, deletions of sequence, single or multiple point mutations, alterations at a particular restriction enzyme recognition site, addition of functional elements, or other means of molecular modification which may enhance, or otherwise alter promoter expression as long as stress-inducibility is essentially retained. Techniques for obtaining such derivatives are well-known in the art (see, for example, J. F. Sambrook, D. W. Russell, and N. Irwin, 2000). For example, one of ordinary skill in the art may delimit the functional elements within the promoters disclosed herein and delete any non-essential elements. The functional elements may be modified or combined to increase the utility or expression of the sequences of the invention for any particular application. Those of skill in the art are familiar with the standard resource materials that describe specific conditions and procedures for the construction, manipulation, and isolation of macromolecules (e.g. DNA molecules, plasmids, etc.), as well as the generation of recombinant organisms and the screening and isolation of DNA molecules.

The promoter sequence of at least 400 consecutive nucleotides of SEQ ID NO: 1 or SEQ ID NO: 2 and their variants as described above may for example be altered to contain e. g. “enhancer DNA” to assist in elevating gene expression. As is well-known in the art, certain DNA elements can be used to enhance the transcription of DNA. These enhancers are often found 5′ to the start of transcription in a promoter that functions in eukaryotic cells, but can often be inserted upstream (5′) or downstream (3′) to the coding sequence. In some instances, these enhancer DNA elements are introns. Among the introns that are useful as enhancer DNA are the 5′ introns from the rice actin 1 gene (see U.S. Pat. No. 5,641,876), the rice actin 2 gene, the Arabidopsis histon 4 intron, the maize alcohol dehydrogenase gene, the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122). Thus, as contemplated herein, a promoter or promoter region includes variations of promoters derived by inserting or deleting regulatory regions, subjecting the promoter to random or site-directed mutagenesis etc. The activity or strength of a promoter may be measured in terms of the amounts of RNA it produces, or the amount of protein accumulation in a cell or tissue, relative to a promoter whose transcriptional activity has been previously assessed, as described above.

As used herein, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S., Chapman & Hall. London, 1995), the homology alignment algorithm of Needleman and Wunsch (1970), the search for similarity method of Pearson and Lipman (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence.

Only first nucleic acids having nucleotide sequences with the above-indicated degree of sequence identity which confer stress inducibility on the chimeric gene described herein are encompassed by the present invention.

In one example, the first nucleic acid as described above comprises the 3′ end of SEQ ID NO: 1 or SEQ ID NO: 2. Said 3′ end comprises at least the last 100 bases, at least the last 200 bases, at least the last 300 bases or at least the last 400 bases of SEQ ID NO: 1 or SEQ ID NO: 2.

In another example, the first nucleic acid sequence comprises at least one of the known response elements selected from the ABA-responsive element (ABRE) from position 796 to 803 in SEQ ID NO: 1 and position 797 to 804 in SEQ ID NO: 2 as well as the two drought-responsive elements DRE1 from position 637 to 645 in SEQ ID NO: 1 and position 637 to 645 in SEQ ID NO: 2 and DRE2 from position 694 to 702 in SEQ ID NO: 1 and position 694 to 702 in SEQ ID NO: 2. In yet another example, said first nucleic acid sequence comprises at least two of the above response elements, such as ABRE and DRE1, ABRE and DRE2 or DRE1 and DRE2. Said first nucleic acid may also comprise all three response elements. In another example, any of the above examples of a first nucleic acid sequence comprising at least one, at least two or all three response elements further comprise the 3′ end of SEQ ID NO: 1 or SEQ ID NO: 2 as described immediately above.

An expression product denotes an intermediate or end product arising from the transcription and optionally translation of the nucleic acid, DNA or RNA, coding for such product. During the transcription process, a DNA sequence under control of regulatory regions, particularly the promoter, is transcribed into an RNA molecule. An RNA molecule may either itself form an expression product and is then, for example, capable of interacting with another nucleic acid or protein. Alternatively, an RNA molecule may be an intermediate product when it is capable of being translated into a peptide or protein. A gene is said to encode an RNA molecule as expression product when the RNA is the end product of the expression of the gene and is capable of interacting with another nucleic acid or protein. Examples of RNA expression products include inhibitory RNA such as e. g. sense RNA (co-suppression), antisense RNA, ribozymes, miRNA or siRNA, mRNA, rRNA and tRNA. A gene is said to encode a protein as expression product when the end product of the expression of the gene is a protein or peptide.

The term “involved in the response of a cotton plant to stress” in connection with expression products of interest indicate that, upon exposure of a plant naturally comprising a gene encoding an expression product as described above to stress conditions, expression of said gene is either switched on or increased, or abolished or decreased, indicating their role in the plant's response to stress. Methods of evaluating whether an expression product is also involved in the response of a cotton plant to stress are known in the art. For example, the reporter gene assay described further above may be employed and the reporter gene may be operably linked to the promoter naturally operably linked to the nucleic acid sequence encoding the expression product. After transformation into cotton plants or cotton plant cells, expression of the reporter gene can be evaluated. If a difference in expression of said reporter gene as compared to one operably linked to a constitutively active promoter, such as that controlling a house-keeping gene, is observed after exposure of the plant or the plant cell to stress, this is indicative that said promoter, and accordingly expression of the nucleic acid encoding the expression product, is inducible by stress. In this regard, it is of note that the expression of such a product does not need to be inducible by all kinds of stress. Rather, it is sufficient that it is inducible by at least one kind of stress applicable to plants as described elsewhere in this application. Another example includes transcriptome analysis of genes involved in stress response, e. g. by applying microarrays.

Confirmation of promoter activity for a promoter sequence or a functional promoter fragment may be determined by those skilled in the art, for example using a promoter-reporter construct comprising the promoter sequence operably linked to an easily scorable marker such as a beta-glucuronidase (GUS) reporter gene as herein further explained. The capability of the identified or generated fragments or variants of the promoter described herein to confer stress inducibility on the chimeric genes they are comprised in can be conveniently tested by operably linking such nucleic acid sequences to a nucleotide sequence encoding an easily scorable marker, e.g. a beta-glucuronidase gene, introducing such a chimeric gene into a plant and analyzing the expression pattern of the marker in upon exposure of the plant to stress as compared with the expression pattern of the marker in plants not exposed to stress. Other candidates for a marker (or a reporter gene) are chloramphenicol acetyl transferase (CAT), beta-galactosidase (beta-GAL), and proteins with fluorescent or phosphorescent properties, such as green fluorescent protein (GFP) from Aequora Victoria or luciferase. To define a minimal promoter, a nucleic acid sequence representing the promoter is operably linked to the coding sequence of a marker (reporter) gene by recombinant DNA techniques well known to the art. The reporter gene is operably linked downstream of the promoter, so that transcripts initiating at the promoter proceed through the reporter gene. The expression cassette containing the reporter gene under the control of the promoter can be introduced into an appropriate cell type by transformation techniques well known in the art and described elsewhere in this application. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography. The level of enzyme activity corresponds to the amount of enzyme that was made, which in turn reveals the level of expression of the promoter or fragment or variant thereof upon stress-exposure of the plant. This level of expression can also be compared to other promoters to determine the relative strength of the promoter under study. Once activity and functionality is confirmed, additional mutational and/or deletion analyses may be employed to determine e. g. a minimal region and/or sequences required to initiate transcription. Thus, sequences can be deleted at the 5′ end of the promoter region and/or at the 3′ end of the promoter region, or within the promoter sequence and/or nucleotide substitutions may be introduced. These constructs are then again introduced into cells and their activity and/or functionality are determined.

Instead of measuring the activity of a reporter enzyme, the transcriptional promoter activity (and functionality) can also be determined by measuring the level of RNA that is produced. This level of RNA, such as mRNA, can be measured either at a single time point or at multiple time points and as such the fold increase can be average fold increase or an extrapolated value derived from experimentally measured values. As it is a comparison of levels, any method that measures mRNA levels can be used. In an example, expression in at least one tissue of a plant exposed to stress is compared with expression in at least one tissue of a plant not exposed to stress. In another example, multiple tissues or organs are compared. As used herein, examples of plant organs are seed, leaf, root, etc. and examples of tissues are leaf primordia, shoot apex, vascular tissue, etc. The activity or strength of a promoter may be measured in terms of the amount of mRNA or protein accumulation it specifically produces, relative to the total amount of mRNA or protein. Alternatively, the activity or strength of a promoter may be expressed relative to a well-characterized promoter (for which transcriptional activity was previously assessed).

Within the scope of the present disclosure, use may also be made, in combination with the first and second nucleic acid sequence described above, of other regulatory sequences, which are located between said first nucleic acid sequence comprising a promoter and said second nucleic acid sequence comprising the coding sequence of the expression product. Non-limiting examples of such regulatory sequences include transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, or introns as described elsewhere in this application. Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression.

The chimeric gene may also comprise a transcription termination or polyadenylation sequence operable in a plant cell, particularly a cotton plant cell. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1.

The nucleotide sequence of SEQ ID NO: 1 represents the promoter of the rd29A gene with one deletion, whereas SEQ ID NO: 2 represents the promoter of the rd29A gene without modification (rd29A is herein under also referred to as rd29). The promoter comprises at least two cis-acting elements one of which is involved in the ABA-associated response (the ABA-responsive element ABRE) to dehydration and the other is induced by changes in osmotic potential.

It has been shown that a nucleic acid sequence SEQ ID NO: 1 corresponding to the rd29 promoter with one base pair deleted is sufficient to activate transcription of operably linked genes in cotton leaves.

Upon transformation into specific plants and subsequent exposure of said plants to various types of abiotic stress a chimeric gene comprising the rd29 promoter operably linked with a heterologous gene could be expressed. This could be shown in transgenic Arabidopsis, tobacco (Yamaguchi-Shinozaki and Shinozaki, 1992, Mol Gen Genet, p: 331-340), potato (Behnam et al, 2007, Plant Cell Rep; p: 1275-1282), Chrysanthemum (Hong et al., 2006, Sci China C Life Sci, p: 436-45) and wheat (Pellegrineschi et al., 2004, Genome, p: 493-500).

Another promoter, the ABA-responsive rice promoter rab l 6A operatively linked to the reporter gene GUS was transformed to tobacco, where no activity of the promoter could be detected in vegetative tissue even after treatment with ABA.

There are examples where promoters transferred to heterologous or, when coupled to a transgene, even homologous plant systems do not necessarily exert their function and expression profile as found in their natural background and operably linked to their natural gene. For example, an ABA-responsive promoter belonging to the Asr family from tomato is shown to be functional and inducible by ABA in its natural background in tomato. However, when coupled to GUS and transformed into potatoes, inducibility abolished. On the other hand, ABA-inducible expression could be observed both in papaya and in tobacco. Surprisingly, the Asr-GUS construct transformed in tomatoes was not inducible by ABA any more, unlike the promoter in its natural genetic context. Accordingly, the behavior of heterologous promoters responsive to stress conditions, in particular stress conditions mediated by ABA is not predictable. In other words, an ABA-responsive promoter functional in one plant does not necessarily exert this function in a transgenic plant.

Aside from this, the abundant concentration of ABA in some plants might lead to a constitutive induction of an ABA-responsive promoter thus preventing a stress-specific response.

The basal concentration of ABA in non-stressed Arabidopsis leaves is 2-3 ng g-1fresh weight (Lopez-Carbonell and Jauregui, 2005). Under drought-stress conditions, the ABA concentration reaches 10-21 ng g-1 fresh weight (f.w.) and activates the promoter of the rd29a gene (the rd29 promoter). However, in non-stressed cotton plants, the ABA concentration in leaves already varies between 145 to 2490 ng g-1 f.w. (Ackerson, 1982). This range of concentrations in cotton would be expected to permanently activate the Arabidopsis rd29 promoter when introduced in cotton. Therefore the use of the rd29 Arabidopsis promoter for drought inducible activation in cotton would not have been considered by the skilled person.

The present inventors generated transgenic cotton plants using the GUS reporter under the control of the Arabidopsis rd29 promoter comprising an ABA-responsive element (ABRE) as well as the two drought-responsive elements DRE1 and DRE2. In the course of the present invention it was surprisingly found that this promoter region triggers GUS expression under water-stress conditions despite the high ABA concentration present in leafs of unstressed cotton plants. Surprisingly, despite the high endogenous level of ABA in cotton leaves, the activity of the rd29 promoter is induced only after drought stress and returns to zero after re-watering.

Furthermore, cotton plants comprising a chimeric gene comprising, as a second nucleic acid, the PNC1 gene, the NMA1 gene or a nucleic acid encoding a micro RNA directed against PARP1 were created which grew well and were fertile, as opposed to plants comprising a chimeric gene comprising the CBF3/CREB1A coding sequence as second nucleic acid (Allen, 2010) under control of rd29. Also PNC1 expression under control of the rd29 promoter was increased in plants exposed to drought stress.

The utility of the chimeric genes described above as well as of the various other aspects disclosed herein will be described below. For example, the disclosure of the present application can be used to modulate the response of a cotton plant to stress, for example in order to facilitate growing cotton plants in regions where cotton plants are exposed to one or more kinds of abiotic stress at least once in their lifetime.

In another aspect, the present application discloses a cotton plant cell comprising a chimeric gene comprising (a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; (b) a second nucleic acid sequence encoding an expression product of interest; and optionally (c) a transcription termination and polyadenylation sequence.

A cotton plant cell may be any cell comprising essentially the genetic information necessary to define a cotton plant, which may, apart from the chimeric gene disclosed herein, be supplemented by one or more further transgenes. Cells may be derived from the various organs and/or tissues forming a cotton plant, including but not limited to fruits, seeds, embryos, reproductive tissue, meristematic regions, callus tissue, leaves, roots, shoots, flowers, vascular tissue, gametophytes, sporophytes, pollen, and microspores.

“Cotton” or “cotton plant” as used herein includes Gossypium hirsutum, Gossypium barbadense, Gossypium arboreum and Gossypium herbaceum or progeny from crosses of such species with other species or crosses between such species.

In one aspect, the cotton plant cell as described above comprises the chimeric gene as described herein.

In one aspect, the cotton plant cell of the invention can be regenerated into a viable and fertile cotton plant (see table 1). Furthermore, the cotton plant described further below is viable and fertile. In other words, plants comprising the chimeric gene of the invention show normal vigor and fertility as compared to wild-type plants.

Whereas certain plant cells according to the invention may be able to regenerate into complete plants, in some embodiments, said plant cells cannot further develop or regenerate into a complete plant.

In one example of the chimeric gene described herein, said expression product of interest is (i) a protein or peptide, or (ii) an RNA molecule capable of modulating the expression of a gene comprised in said cotton plant. Said protein or peptide or said gene comprised in said cotton plant is preferably involved in the response of a cotton plant to stress. A gene comprised in a cotton plant may be endogenous to the cotton plant or have been introduced into said cotton plant. The latter in particular applies to target expression products which are not endogenous to cotton plants or to homologs of expression products endogenous in cotton plants from other organisms, but which are involved in the response of a cotton plant to stress.

In one example of the cotton plant cell as described herein, said expression product of interest is (i) a protein or peptide, optionally involved in the response of a cotton plant to stress or (ii) an RNA molecule capable of modulating the expression of a gene comprised in said cotton plant, wherein optionally said gene is involved in the response of a cotton plant to stress.

The term “protein” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

Example proteins and nucleic acids, such as genes, optionally involved in the response of a cotton plant to stress, which are suitable as expression products include:

NPT1 (nicotinate phosphoribosyltransferase) which acts in the salvage pathway of NAD⁺ biosynthesis, and the gene encoding it. The protein is required for silencing at rDNA and telomeres and has a role in silencing at mating-type loci. As for all other genes suitable in the present invention, the sequences encoding NPT1 which can be used in the present invention may be from animal, plant or fungal origin. Exemplary nucleic acid sequences encoding NPT1 encode amino acid sequences including those having accession number CAA85352 (Saccharomyces cerevisae), XP 448893 (Candida glabrata), XP_453357 (Kluyveromyces lactis), NP_983562 (Eremothecium gossypii), XP_462577 (Debaromyces hansenii), XP_889008 (Candida albicans), XP_500338 (Yarrowia lipolytica), XP_746744 (Aspergillus fumigatus), BAE64333 (Aspergillus oryzae), XP_965789 (Neurospora crassa), EAQ93453 (Chaetomium globosum), XP_682385 (Aspergillus nidulans), AAN74808 (Gibberella moniliformis), Q9UTK3, XP_361075 (Magnaporthe grisea), EAL18922 (Cryptococcus neoformans), XP_568039 (Cryptococcus neoformans) and XP_760597 (Ustilago maydis). The S. cerevisiae NPTI complete cDNA and encoded protein are provided by GenBank Accession numbers NC_001147 and AAB59317, respectively. The E. coli NPT1 is provided as GenBank accession number J05568. The human nucleotide and amino acid sequences are provided by GenBank Accession numbers BC006284 and AAH06284, respectively, and X71355 and CAA50490, respectively, AAH32466 and BC032466 and are described in Chong et al. (1993) Genomics 18:355. Mouse NPT1 nucleotide and amino acid sequences are provided by GenBank Accession numbers X77241 and CAA54459 and are described in Chong et al. (1995) Am. J. Physiol. 5 268: 1038.

PNC1 (pyrazinamidase/nicotinamidase 1), a nicotinamidase that converts nicotinamide to nicotinic acid as part of the NAD salvage pathway, and the gene encoding it. The enzyme is required for life span extension by calorie restriction. Exemplary nucleic acid sequences encoding PNC1 encode amino acid sequences including those having accession number Q06178, XP_444815 (Candida glabrata), NP_986687 (Eremothecium gossypii), XP_453005 (Kluyveromyces lactis), XP_458184 (Debaromyces hansenii), XP_718656 (Candida albicans), XP_504391 (Yarrowia lipolytica), NP_592856 (Schizosaccharomyces pombe), XP_762639 (Ustilago maydis), XP_571297 (Cryptococcus neoformans), BAE57070 (Aspergillus oryzae), XP_750776 (Aspergillus fumigatus), XP_659349 (Aspergillus nidulans), XP_389652 (Giberella zeae), XP_957634 (Neurospora crassa), XP_363364 (Magnaporthe grisea), XP_758179 (Ustilago maydis) and EAQ85219 (Chaetomium globosum). A nucleotide sequence encoding S. cerevisiae PNC1 and the protein encoded thereby are represented in GenBank Accession numbers NC_001139 and NP_011478, respectively. The nucleotide and amino acid sequences of an Arachis hypogaea PNC1 are provided by GenBank Accession numbers M37636 and AAB06183 and are described in Buffard et al. (1990) PNAS 87:8874. Nucleotide and amino acid sequences of a human homolog are provided by GenBank Accession numbers BCOI7344 and AAH17344, respectively; AK027122 and NP_078986, respectively; XM_041059 and XP_041059, respectively; and NM_016048 and NP_057132, respectively. The nucleotide and amino acid sequences of human PNC1 are represented in GenBank Accession No. BC017344.

NMA1 (nicotinic acid mononucleotide adenylyltransferase 1), involved in NAD⁺ salvage pathway, and the gene encoding it. Exemplary nucleic acid sequences encoding NMA1 encode amino acid sequences including those having accession number Q06178, XP_444815 (Candida glabrata), NP_986687 (Eremothecium gossypii), XP_453005 (Kluyveromyces lactis), XP_458184(Debaromyces hansenii), XP_718656 (Candida albicans), XP_504391 (Yarrowia lipolytica), NP_592856 (Schizosaccharomyces pombe), XP_762639 (Ustilago maydis), XP_571297 (Cryptococcus neoformans), BAE57070 (Aspergillus oryzae), XP_750776 (Aspergillus fumigatus), XP_659349 (Aspergillus nidulans), XP_389652 (Giberella zeae), XP_957634 (Neurospora crassa), XP_363364 (Magnaporthe grisea), XP_758179 (Ustilago maydis) and EAQ85219 (Chaetomium globosum). A nucleotide sequence encoding S. cerevisae NMA1 and the protein encoded thereby are represented in GenBank Accession Numbers NC_001144.2 and NP_013432, respectively. Nucleotide and amino acid sequences of human homologs are provided by GenBank Accession numbers NM_022787 and NP_073624, respectively; AK026065 and BAB15345, respectively; AF459819 and AAL76934, respectively; XM_087387 and XP_087387, respectively; and AF345564 and AAK52726, respectively, and NP_073624; AAL76934; NP_073624; and AF314163. Bacterial homologs are described, e.g., in Zhang et aL (2002) Structure 10:69.

NMA2 (nicotinic acid mononucleotide adenylyltransferase 2), involved in de novo and salvage synthesis of NAD⁺, and the gene encoding it.

For examples for the above four proteins and the genes encoding them, see also WO2006/032469. Exemplary nucleic acid sequences encoding NMA2 encode amino acid sequences including those having accession number NP_011524, XP_444815 (Candida glabrata), NP_986687 (Eremothecium gossypii), XP_453005 (Kluyveromyces lactis), XP_458184 (Debaromyces hansenii), XP_718656 (Candida albicans), XP_504391 (Yarrowia lipolytica), NP_592856 (Schizosaccharomyces pombe), XP_762639 (Ustilago maydis), XP_571297 (Cryptococcus neoformans), BAE57070 (Aspergillus oryzae), XP_750776 (Aspergillus fumigatus), XP_659349 (Aspergillus nidulans), XP_389652 (Giberella zeae), XP_957634 (Neurospora crassa), XP_363364 (Magnaporthe grisea), XP_758179 (Ustilago maydis) and EAQ85219 (Chaetomium globosum). A nucleotide sequence encoding S. cerevisiae NMA2 and the protein encoded thereby are represented in GenBank Accession numbers NC_001139 and NP_011524, respectively. Nucleotide and amino acid sequences of human homologs are provided by GenBank Accession numbers NM_015039 and NP_055854, respectively. A nucleotide sequence encoding S. cerevisiae NMA2 and the protein encoded thereby are represented in GenBank Accession numbers NC_001139 and NP_011524, respectively. Nucleotide and amino acid sequences of human homologs are provided by GenBank Accession numbers NM_015039 and NP_055854, respectively.

Proteins involved in oxidative stress such as choline oxidase (COD), superoxide dismutase (SOD) and ascorbate peroxidase (APX), and the genes encoding them. (Ahmad et al., 2010).

Transcription factors, including G1073 (atHRCI), and equivalogs in the G1073 Glade of transcription factor polypeptides as disclosed in EP1668140.

Los5, a key regulator of ABA biosynthesis, involved in stress-responsive gene expression, and stress tolerance (Xiong et al., The Plant Cell (2001), Vol. 13, 2063-2083), and the gene encoding it.

Any gene encoding an expression product of interest may be endogenous to cotton plants or may have been introduced into a cotton plant. In the latter case, the gene introduced may be either a gene homologs of which are not found in cotton or one which has a homolog in cotton. For example, a gene encoding NPT1 may be derived from fungi such as yeast.

Said expression product of interest may also be an RNA molecule capable of modulating the expression of a gene comprised in said cotton plant, wherein said gene is optionally involved in the response of a cotton plant to stress.

Examples of genes involved in the response of a cotton plant to stress include PARP1, PARP2, FTA, FTB, NPT1, PNC1, NMA1, NMA2 and Los5.

FTA (farnesytransferase alpha) and FTB (farnesytransferase beta) are signaling genes identified as playing a role in a plant's ability to respond to environmental stresses such as drought (see also Wang et al., 2005). Farnesyl transferase catalyses the first step of farnesylation in which a 15-carbon farnesyl moiety is added to the cysteine residue of the target sequence CaaX. Example uses of FTA and FTB-related expression products can also be found in EP1534842.

For the case of RNA molecules, it will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

The term “capable of modulating the expression of a gene” relates to the action of an RNA molecule, such as an inhibitory RNA molecule as described herein, to influence the expression level of target genes in different ways. This can be effected by inhibiting the expression of a target gene by directly interacting with components driving said expression such as the gene itself or the transcribed mRNA which results in a decrease of expression, or another gene involved in inhibiting the expression of a gene, wherein said latter gene is optionally involved in the response of a cotton plant to stress, which results in an increase of expression.

Inhibitory RNA molecules decrease the levels of mRNAs of their target expression products such as target proteins available for translation into said target protein. In this way, expression of proteins, for example those involved in unwanted responses to stress conditions, can be inhibited. This can be achieved through well established techniques including co-suppression (sense RNA suppression), antisense RNA, double-stranded RNA (dsRNA), or microRNA (miRNA).

An RNA molecule as expression product as disclosed herein comprises a part of a nucleotide sequence encoding a target expression product such as target protein or RNA or a homologous sequence to down-regulate the expression of said target expression product. Another example for an RNA molecule as expression product for use in down-regulating expression are antisense RNA molecules comprising a nucleotide sequence complementary to at least a part of a nucleotide sequence encoding an expression product such as a protein or RNA of interest or a homologous sequence. Here, down-regulation may be effected e. g. by introducing this antisense RNA or a chimeric DNA encoding such RNA molecule. In yet another example, expression of an expression product of interest such as a protein or RNA of interest is down-regulated by introducing a double-stranded RNA molecule comprising a sense and an antisense RNA region corresponding to and respectively complementary to at least part of a gene sequence encoding said expression product of interest, which sense and antisense RNA region are capable of forming a double stranded RNA region with each other. Such double-stranded RNA molecule may be encoded both by sense and antisense molecules as described above and by a single-stranded molecule being processed to form siRNA (as described e. g. in EP1583832) or miRNA.

In one example, expression of a target protein may be down-regulated by introducing a chimeric DNA construct which yields a sense RNA molecule capable of down-regulating expression by co-suppression. The transcribed DNA region will yield upon transcription a so-called sense RNA molecule capable of reducing the expression of a gene encoding a target expression product such as a target protein or RNA in the target plant or plant cell in a transcriptional or post-transcriptional manner. The transcribed DNA region (and resulting RNA molecule) comprises at least 20 consecutive nucleotides having at least 95% sequence identity to the corresponding portion of the nucleotide sequence encoding the target expression product such as a target protein present in the plant cell or plant.

Alternatively, an expression product for down-regulating expression of a target expression product such as a target protein or RNA is an antisense RNA molecule.

Down-regulating or reducing the expression of an expression product of interest in the target cotton plant or plant cell is effected in a transcriptional or post-transcriptional manner. The transcribed DNA region (and resulting RNA molecule) comprises at least 20 consecutive nucleotides having at least 95% sequence identity to the complement of the corresponding portion of the nucleic acid sequence encoding said target expression product present in the plant cell or plant.

However, the minimum nucleotide sequence of the antisense or sense RNA region of about 20 nt of the nucleic acid sequence encoding a target expression product may be comprised within a larger RNA molecule, varying in size from 20 nt to a length equal to the size of the target gene. The mentioned antisense or sense nucleotide regions may thus be about from about 21 nt to about 5000 nt long, such as 21 nt, 40 nt, 50 nt, 100 nt, 200 nt, 300 nt, 500 nt, 1000 nt, 2000 nt or even about 5000 nt or larger in length. Moreover, it is not required for the purpose of the invention that the nucleotide sequence of the used inhibitory RNA molecule or the encoding region of the transgene, is completely identical or complementary to the target gene, which may be endogenous to the plant or have been introduced, encoding the target expression product the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the target gene or the complement thereof. However, as mentioned, antisense or sense regions should comprise a nucleotide sequence of 20 consecutive nucleotides having about 95 to about 100% sequence identity to the nucleotide sequence encoding the target gene. The stretch of about 95 to about 100% sequence identity may be about 50, 75 or 100 nt.

The efficiency of the above mentioned chimeric genes for antisense RNA or sense RNA-mediated gene expression level down-regulation may be further enhanced by inclusion of DNA elements which result in the expression of aberrant, non-polyadenylated inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

In addition, an expression product as described herein may be a nucleic acid sequence which yields a double-stranded RNA molecule capable of down-regulating expression of a gene encoding a target expression product. Upon transcription of the DNA region the RNA is able to form dsRNA molecule through conventional base paring between a sense and antisense region, whereby the sense and antisense region are nucleotide sequences as hereinbefore described. Expression products being dsRNA according to the invention may further comprise an intron, such as a heterologous intron, located e.g. in the spacer sequence between the sense and antisense RNA regions in accordance with the disclosure of WO 99/53050. To achieve the construction of such a transgene, use can be made of the vectors described in WO 02/059294 A1.

In an example, said RNA molecule comprises a first and second RNA region wherein 1. said first RNA region comprises a nucleotide sequence of at least 19 consecutive nucleotides having at least about 94% sequence identity to the nucleotide sequence of said endogenous gene; 2. said second RNA region comprises a nucleotide sequence complementary to said 19 consecutive nucleotides of said first RNA region; 3. said first and second RNA region are capable of base-pairing to form a double stranded RNA molecule between at least said 19 consecutive nucleotides of said first and second region.

Another example expression of interest product is a microRNA molecule (mirRNA, which may be processed from a pre-microRNA molecule) capable of guiding the cleavage of mRNA transcribed from the DNA encoding the target expression product, such as a protein or an RNA, which is to be translated into said target expression product. miRNA molecules or pre-miRNA molecules may be conveniently introduced into plant cells through expression from a chimeric gene as described herein comprising a (second) nucleic acid sequence encoding as expression product of interest such miRNA, pre-miRNA or primary miRNA transcript.

miRNAs are small endogenous RNAs that regulate gene expression in plants, but also in other eukaryotes. As used herein, a “miRNA” is an RNA molecule of about 19 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule.

In one example, one or more of the following mismatches may occur in the essentially complementary sequence of the miRNA molecule:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches;
No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* does not need to be perfect and about 1 to 3 bulges of unpaired nucleotides can be tolerated. The secondary structure adopted by an RNA molecule can be predicted by computer algorithms conventional in the art such as mFold, UNAFoId and RNAFold. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bonding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

Example expression products can also be ribozymes catalyzing either their own cleavage or the cleavage of other RNAs.

In one example of the chimeric gene disclosed herein modulating is increasing and said second nucleic acid sequence encodes an RNA, which when transcribed 1. yields an RNA molecule capable of increasing the expression of a gene endogenous to said cotton plant, said gene being selected from NPT 1, PNC 1, Los5, NMA 1 and NMA2, e. g. by targeting genes involved in down-regulating the expression of these proteins, or 2. yields an RNA molecule capable of decreasing the expression of a gene endogenous to said cotton plant, said gene being selected from PARP1, PARP2, FTA and FTB, for example by targeting this gene directly.

In another example of the chimeric gene disclosed herein modulating is decreasing and said second nucleic acid sequence encodes an RNA, which when transcribed 1. yields an RNA molecule capable of increasing the expression of a gene endogenous to said cotton plant, said gene being selected from PARP1, PARP2, FTA and FTB, e. g. by targeting genes involved in down-regulating the expression of these proteins, or 2. yields an RNA molecule capable of decreasing the expression of a gene endogenous to said cotton plant, said gene being selected from NPT 1, PNC 1, Los5, NMA 1 and NMA2, for example by targeting this gene directly.

Example RNA-based expression products include inhibitory RNAs such as miRNAs, siRNAs, antisense RNAs or ribozymes targeting enzymes of the PARP (poly(ADP-ribose) polymerase) family, examples of which are also disclosed in international patent application PCT/EP2010/003438.

Currently, two classes of PARP proteins have been described. The first class, as defined herein, comprises the so-called classical Zn-finger containing PARP proteins (ZAP), or PARP1 proteins, encoded by corresponding parp1 genes. These proteins range in size from 113-120 kDa and are further characterized by the presence of at least one, preferably two Zn-finger domains located in the N-terminal domain of the protein, particularly located within the about 355 to about 375 first amino acids of the protein. The Zn-fingers are defined as peptide sequences having the sequence CxxCxnHxxC (whereby n may vary from 26 to 30) capable of complexing a Zn atom. Examples of amino acid sequences for PARP proteins from the ZAP class which can be used as a basis for designing expression products in accordance with the present invention include the sequences which can be found in the PIR protein database with accession number P18493 (Bos taurus), P26466 (Gallus gallus), P35875 (Drosophila melanogaster), P09874 (Homo sapiens), P11103 (Mus musculus), Q08824 (Oncorynchus masou), P27008 (Rattus norvegicus), Q11208 (Sarcophaga peregrina), and P31669 (Xenopus laevis). The nucleotide sequence of the corresponding cDNAs can be found in the EMBL database under accession numbers D90073 (Bos taurus), X52690 (Gallus gallus), D13806 (Drosophila melanogaster), M32721 (Homo sapiens), X14206 (Mus musculus), D13809 (Oncorynchus masou), X65496 (Rattus norvegicus), D16482 (Sarcophaga Peregrina), and D14667 (Xenopus laevis). PARP1 proteins have been described in maize (WO 00/04173). In Arabidopsis thaliana, a parp1 gene with AGI number At2g31320 is reported in the TAIR8 protein database.

The second class as defined herein, comprises the so-called non-classical PARP proteins (NAP) or PARP2 proteins, encoded by corresponding parp2 genes. These proteins are smaller (72-73 kDa) and are further characterized by the absence of a Zn-finger domain at the N-terminus of the protein, and by the presence of an N-terminal domain comprising stretches of amino acids having similarity with DNA binding proteins. PARP2 proteins have been reported in maize (WO 00/04173) and in cotton (WO 2006/045633). Two parp2 genes have been identified in the genome of Arabidopsis thaliana (At4g02390 and At5g22470).

The following is a non-limiting list of database entries identifying experimentally demonstrated and putative plant PARP protein sequences that could be identified and that can be taken as a basis for designing expression products according to the invention: AAN12901, AAM13882, CAA10482, AAD20677, BAB09119, CAB80732, CAA88288, AAC19283, Q9ZP54, Q9FK91, Q11207, NP_850165, NP_197639, NP_192148 (Arabidopsis thaliana); CAO70689, CAN75718, CAO48763, CAO40033, A7QVS5, A5AIW8, A7Q0E8, A5AUF8, A7QFD4 (Vitis vinifera); BAF21367, BAC84104, EAZ03601, EAZ39513, BAF08935, EAZ23301, EAY86124, BAD25449, BAD53855, BAD52929, EAZ11816, BAF04898, BAF04897, EAY73948, EAY73947, EAZ11816, EAZ11815, Q7EYV7, Q0E003, A2YKJO, A2X5L4, A2WPQ2, A2WPQ1, A3BIX4, A3A7L2, A2ZSW9, Q5Z8Q9, Q0JMY1, A2ZSW8, NP_001059453, NP_001047021, NP_001042984, NP_001042983 (Oryza sativa); AAC79704, CAA10889, CAA10888, Q9ZSV1, O50017, B4FCJ3 (Zea mays); EDQ65830, EDQ52960, A9SSX0, A9TUE0, A9S9 P7 (Physcomitrella patens); AAD51626, Q9SWB4 (Glycine max), Q1SGF1 (Medicago truncatula); ABK93464, A9PAR1 (Populus trichocarpa).

It is clear that other genes or cDNAs encoding PARP1 or PARP2 proteins, or parts thereof, can be isolated from other eukaryotic species or varieties, particularly from other plant species or varieties. Moreover, parp1 or parp2 genes, encoding PARP1 proteins wherein some of the amino acids have been exchanged for other, chemically similar, amino acids (so-called conservative substitutions), or synthetic parp1 genes (which encode similar proteins as natural parp1 genes but with a different nucleotide sequence, based on the degeneracy of the genetic code) and parts thereof are also suited for the methods of the invention.

In one example of the chimeric gene and the cotton plant cell described herein said first nucleic acid sequence comprises the nucleotide sequence of SEQ ID NO: 1 or SEQ ID NO: 2 or a nucleic acid sequence having at least 70%, at least 80%, at least 90%, at least 95% or at least 98% sequence identity thereto and conferring stress inducibility on said chimeric gene.

In another example of the chimeric gene and the cotton plant cell described herein said first nucleic acid sequence consists of SEQ ID NO: 1 or SEQ ID NO: 2 or a nucleic acid sequence having at least 70%, at least 80%, at least 90%, at least 95% or at least 98% sequence identity thereto and conferring stress inducibility on said chimeric gene.

In one example of the chimeric gene and the cotton plant cell disclosed herein said stress is water stress, cold stress, high-salt stress or the application of ABA.

These stress factors are summarized under the term “abiotic plant environmental stress”. These factors are not isolated but also interrelated and influencing each other.

Water stress includes drought resulting in a shortage of water for the plant.

Drought is one of the most serious world-wide problems for agriculture. Four-tenths of the world's agricultural land lies in arid or semi-arid regions. Transient droughts can cause death of livestock, famine and social dislocation. Other agricultural regions have consistently low rain-fall and rely on irrigation to maintain yields. In both circumstances, crop plants which can make the most efficient use of water and maintain acceptable yields will be at an advantage.

It has been shown in the examples of this application that a transgene or chimeric gene can be efficiently expressed under the control of the rd29 promoter in cotton plant cells upon exposure to drought stress. This enables for alleviating the effect of drought conditions for the plant by providing sequences encoding expression products which reduce the shortcomings related thereto, such as for example described for expression products decreasing the expression of PARP.

Drought as used in the present application relates to the shortage or absence of water available to a plant for a specified time. Such shortage or absence of water may last only a few days such as at least or up to 2, at least or up to 3, at least or up to 4, at least or up to 5, at least or up to 6, at least or up to 7, at least or up to 8, at least or up to 9, at least or up to 10, at least or up to 15 or at least or up to 20 days. It may as well be for a longer period such as at least or up to 3 weeks, at least or up to 4 weeks, at least or up to 5 weeks, at least or up to 6 weeks, at least or up to 2 months, at least or up to 3 months, at least or up to 4 months, at least or up to 5 months or at least or up to 6 months. In some areas of the world, drought may even last longer than 6 month, such as 7, 8, 9, 10, 11, 12, 15, 18 or 24 months.

The term “cold stress” in connection with the present application denotes a temperature of less than 12° C., less than 11° C., less than 10° C., less than 9° degrees, less than 8° C., less than 7° C., less than 6° C., less than 5° C., less t han 4° C., less than 3° C., less than 2° C., less than 1° C. or even less than 0° C. such as less th an −2° C., less than −4° C., less than −6° , less than −8° C., less than −10° C. such as −15° C., −20° C. or −25° C. for a specified period of time such as at least 5 h, at least or up to 6 h (the term “up to” in connection with this aspect also including “at least 5 h), at least 7 h, at least 8 h, at least 9 h, at least 10 h, at least 15 h, at least 20 h, at least 1 day, at least 2 days, at least 3 days, at least 4 days, at least 5 days, at least 6 days, at least 7 days, at least 10 days, at least 14 days, at least 21 days or at least 28 days. Any combination of the above two lists adequately define cold stress. For example, cold stress is present at a temperature of less than 10′C for at least 6 h, at least 12 h, at least 1 day, at least 2 days, at least 4 days, at least 1 week or at least 2 weeks.

Salt stress has been reported to cause an inhibition of growth and development, reduction in photosynthesis, respiration and protein synthesis in sensitive species (Boyer, 1982; Meloni et al., 2003; Pal et al., 2004). An important consequence of salinity stress in plants is the excessive generation of reactive oxygen species (ROS) such as superoxide anion (O⁻²), hydrogen peroxide (H₂O₂) and the hydroxyl radicals (OH.) particularly in chloroplasts and mitochondria (Mittler, 2002; Masood et al., 2006). The term “high-salt stress” denotes a salt concentration in the soil surrounding a plant, in particular a cotton plant, of at least 80 mM, at least 90 mM, at least 100 mM, at least 120 mM, at least 130 mM, at least 140 mM, at least 150 mM or at least 200 mM. The kind of salt may vary depending on the areas and the soils found therein. Exemplary salts are NaCl, CaCl₂, MgCl₂ and MgSO₄.

The application of ABA may be used in experimental setups to mimic abiotic environmental stress since ABA triggers the expression of drought inducible genes. In this regard, the plants may for example be sprayed with solutions comprising an appropriate concentrations of ABA which ranges from at least 20 μM to 500 μM. Plant can also be grown on solid medium containing 50 μM ABA (Hongxia Liu, Plant Cell 2010).

In another aspect, the present application discloses a cotton plant or seed thereof or cotton plant part comprising (a) a chimeric gene comprising a. a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; b. a second nucleic acid sequence encoding an expression product of interest; and optionally c. a transcription termination and polyadenylation sequence; or (b) the cotton plant cell described herein. The chimeric gene described in (a) may be the chimeric gene as described herein above including all variations related thereto.

The chimeric gene may be introduced by transformation in cotton plants from which embryogenic callus can be derived, such as Coker 312, Coker310, Coker 5Acala SJ-5, GSC25110, FIBERMAX 819 , Siokra 1-3, T25, GSA75, Acala SJ2, Acala SJ4, Acala SJ5, Acala SJ-C1, Acala B1644, Acala B1654-26, Acala B1654-43, Acala B3991, Acala GC356, Acala GC510, Acala GAM1, Acala C1, Acala Royale, Acala Maxxa, Acala Prema, Acala B638, Acala B1810, Acala B2724, Acala B4894, Acala B5002, non Acala “picker” Siokra, “stripper” variety FC2017, Coker 315, STONEVILLE 506, STONEVILLE 825, DP50, DP61, DP90, DP77, DES119, McN235, HBX87, HBX191, HBX107, FC 3027, CHEMBRED A1, CHEMBRED A2, CHEMBRED A3, CHEMBRED A4, CHEMBRED B1, CHEMBRED B2, CHEMBRED B3, CHEMBRED C1, CHEMBRED C2, CHEMBRED C3, CHEMBRED C4, PAYMASTER 145, HS26, HS46, SICALA, PIMA S6 ORO BLANCO PIMA, FIBERMAX FM5013, FIBERMAX FM5015, FIBERMAX FM5017, FIBERMAX FM989, FIBERMAX FM832, FIBERMAX FM966, FIBERMAX FM958, FIBERMAX FM989, FIBERMAX FM958, FIBERMAX FM832, FIBERMAX FM991, FIBERMAX FM819, FIBERMAX FM800, FIBERMAX FM960, FIBERMAX FM966, FIBERMAX FM981, FIBERMAX FM5035, FIBERMAX FM5044, FIBERMAX FM5045, FIBERMAX FM5013, FIBERMAX FM5015, FIBERMAX FM5017 or FIBERMAX FM5024 and plants with genotypes derived thereof.

Seed is formed by an embryonic plant enclosed together with stored nutrients by a seed coat. It is the product of the ripened ovule of gymnosperm and angiosperm plants, to the latter of which cotton belongs, which occurs after fertilization and to a certain extent growth within the mother plant.

The transformed cotton plant cells and cotton plants disclosed herein or obtained by the methods described herein may contain, in addition to the chimeric gene described above, at least one other chimeric gene comprising a nucleic acid encoding an expression product of interest. Examples of such expression product include RNA molecules or proteins, such as for example an enzyme for resistance to a herbicide, such as the bar or pat enzyme for tolerance to glufosinate-based herbicides (EP 0 257 542, WO 87/05629 and EP 0 257 542, White et al. 1990), the EPSPS enzyme for tolerance to glyphosate-based herbicides such as a double-mutant corn EPSPS enzyme (U.S. Pat. No. 6,566,587 and WO 97/04103), or the HPPD enzyme for tolerance to HPPD inhibitor herbicides such as isoxazoles (WO 96/38567).

The transformed plant cells and plants obtained by the methods described herein may be further used in breeding procedures well known in the art, such as crossing, selfing, and backcrossing. Breeding programs may involve crossing to generate an F1 (first filial) generation, followed by several generations of selfing (generating F2, F3, etc.). The breeding program may also involve backcrossing (BC) steps, whereby the offspring is backcrossed to one of the parental lines, termed the recurrent parent. Accordingly, also disclosed herein is a method for producing plants comprising the chimeric gene disclosed herein comprising the step of crossing the cotton plant disclosed herein with another plant or with itself and selecting for offspring comprising said chimeric gene.

The transformed plant cells and plants obtained by the methods disclosed herein may also be further used in subsequent transformation procedures, e. g. to introduce a further chimeric gene.

The cotton plants or seed comprising the chimeric gene disclosed herein or obtained by the methods disclosed herein may further be treated with cotton herbicides such as Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron; cotton insecticides such as Acephate, Aldicarb, Chlorpyrifos, Cypermethrin, Deltamethrin, Abamectin, Acetamiprid, Emamectin Benzoate, Imidacloprid, lndoxacarb, Lambda-Cyhalothrin, Spinosad, Thiodicarb, Gamma-Cyhalothrin, Spiromesifen, Pyridalyl, Flonicamid, Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor; and cotton fungicides such as Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin.

In another aspect, disclosed is a method of expressing a transgene in cotton under stress conditions comprising: (a1) introducing or introgressing a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene, a second nucleic acid sequence encoding an expression product of interest, and optionally a transcription termination and polyadenylation sequence into a cotton plant and growing the plant; or (a2) growing the cotton plant described herein or growing a plant from the seed described herein; (b) having said plant exposed to stress. The chimeric gene described in (a1) may be the chimeric gene as described herein above including all variations related thereto.

“Introducing” in connection with the present application relates to the placing of genetic information in a plant cell or plant by artificial means. This can be effected by any method known in the art for introducing RNA or DNA into plant cells, tissues, protoplasts or whole plants.

A number of methods are available to transfer DNA into plant cells. Agrobacterium-mediated transformation of cotton has been described e.g. in U.S. Pat. No. 5,004,863, in U.S. Pat. No. 6,483,013 and WO2000/71733.

Plants may also be transformed by particle bombardment: Particles of gold or tungsten are coated with DNA and then shot into young plant cells or plant embryos. This method also allows transformation of plant plastids. Cotton transformation by particle bombardment is reported e.g. in WO 92/15675.

Viral transformation (transduction) may be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004.

“Introgressing” means the integration of a gene in a plant's genome by natural means, i. e. by crossing a plant comprising the chimeric gene described herein with a plant not comprising said chimeric gene. The offspring can be selected for those comprising the chimeric gene.

Further transformation and introgression protocols can also be found in U.S. Pat. No. 7,172,881.

In the course of expressing the transgene of choice encoded by the chimeric gene disclosed herein, the plant has to be exposed to stress conditions, either naturally or artificially generated. This includes exposing the plant to at least one kind of abiotic environmental stress, such as water stress, in particular drought stress, cold stress, high-salt stress or stress induced by the application of ABA.

Water stress in the form of drought stress may be applied to the plant simply by depriving it of or reducing its water supply, either by placing them in a naturally drought exposed region or by reducing water supply in the greenhouse or in the field. For example, the water supply may be reduced by at least 10%, at least 20%, at least 30%, at least 40%, at least 50%, at least 60%, at least 70%, at least 80%, at least 90% or even 100% for a desired time falling within those described above in connection with drought stress.

Cold stress may be applied by placing the plant at a lower temperature than it is used to. For example, the cotton plant may be placed at a temperature lower than 12′C, lower than 10° C., lower than 7° C. or even as low as 4° C. or 2° C. for a desired time falling within those described above in connection with cold stress.

High-salt stress may be applied by placing the plant in soil which comprises a total salt concentration for a desired time as described above for high-salt stress or by watering the plant with water comprising a salt concentration leading to enrichment of salt in the soil. Exemplary concentrations range between 25 mM and 200 mM, for example between 30 and 180 mM, between 50 and 150 mM, including 60 mM, 70 mM, 80 mM, 90 mM, 100 mM, 110 mM, 120 mM, 130 mM and 140 mM.

Stress induced by ABA may be applied by spraying the plants with a solution comprising ABA at a concentration of between about 10 and about 200 μM, such as between 20 and 150 or 50 to 100.

In a further aspect, the present application discloses a method of producing a cotton plant comprising: introducing or introgressing a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene, a second nucleic acid sequence encoding an expression product of interest, and optionally a transcription termination and polyadenylation sequence; or growing the plant described herein or growing a plant from the seed disclosed herein. The chimeric gene introduced or introgressed may be the chimeric gene as described herein above including all variations related thereto. The expression product of interest encoded by said second nucleic acid sequence comprised in said chimeric gene may be involved in the response of a cotton plant to stress as described above.

Also disclosed herein is a method of detecting the expression of a transgene under stress conditions, comprising (a) providing the cotton plant cell or the plant disclosed herein, wherein said expression product of interest is the transgene; (b) having the plant exposed to stress; and (c) detecting the expression of the transgene.

The term “expression of a transgene” relates to the transcription and optionally the translation of the chimeric gene disclosed herein using appropriate expression control elements that function in cotton cells. As described above, the first nucleic acid sequence disclosed herein has promoter function and is inducible by abiotic plant stress and is thus suitable to express an expression product of choice (corresponding to the second nucleic acid sequence) in cotton under stress conditions.

The exposition of a plant to stress in connection with this method may be effected as described above.

“Detecting the expression of the transgene” can be effected in multiple ways. In case of the transgene being a reporter gene, expression of said reporter gene, depending on the feature rendering it a reporter gene, is easily detectable. For example if the reporter gene is an enzyme capable of converting a substrate into a visually detectable product, said product may be detected by the appropriate means which depend on the color of said product or of the wavelength of the light emitted by said product. In case the transgene is not a conventional reporter gene but has enzymatic activity, assays can be designed by the skilled person knowing said enzymatic activity to track and quantify it with suitable methods. Furthermore, expression of a transgene with known nucleic acid sequence can be measured by PCR methods including the one disclosed in Zanoni et al. (Nature 2009, 460, p:264-269, see also Nature Protocols: mRNA expression analysis by Real-Time PCR; ISSN: 1754-2189) and in Logan, Edwards and Saunders (Editors; Real-Time PCR: Current Technology and Applications, Caister Academic Press 2009, ISBN: 978-1-904455-39-4), by sequencing techniques including that disclosed in the Illumina datasheet “mRNA expression analysis” (2010) available at http://www.illumina.com/documents/products/datasheets/datasheet_mrna_expression.pdf, and by hybridization techniques such as that disclosed in Chaudhary et al. (EVOLUTION & DEVELOPMENT 2008; 10:5, 567-582).

Also disclosed herein is a method for modulating the resistance of a cotton plant to stress comprising introducing or introgressing into a cotton plant a chimeric gene comprising a. a first nucleic acid sequence comprising at least 400 consecutive nucleotides of SEQ ID NO: 1 or SEQ ID NO: 2 or a nucleic acid sequence having at least 80% sequence identity thereto, any of which confers stress inducibility on said chimeric gene; b. a second nucleic acid sequence encoding an expression product of interest which is optionally involved in the response of a cotton plant to stress; and optionally c. a transcription termination and polyadenylation sequence; and having said chimeric gene expressed under stress conditions. The chimeric gene utilized in this method may be the chimeric gene as described herein above including all variations related thereto.

A plant's resistance to stress may be modified upon expression of the chimeric gene described herein under stress conditions if the expression product encoded by the second nucleic acid sequence of said chimeric gene is involved the response of a cotton plant to abiotic environmental stress as described above.

In all methods described herein stress is to be interpreted as above in connection with the chimeric gene disclosed herein. Accordingly, in the methods disclosed herein said stress is water stress, cold stress, high-salt stress or the application of ABA. The water stress may be drought stress.

In one example of the method for modulating the resistance of a cotton plant to stress, modulating is increasing and said second nucleic acid sequence encodes an RNA, which when transcribed 1. yields an RNA molecule capable of increasing the expression of a gene comprised in said cotton plant, said gene being selected from NPT 1, PNC 1, Los5, NMA 1 and NMA2, e. g. by targeting genes involved in down-regulating the expression of these proteins, or 2. yields an RNA molecule capable of decreasing the expression of a gene comprised in said cotton plant, said gene being selected from PARP1, PARP2, FTA and FTB, for example by targeting this gene directly.

In another example of the method for modulating the resistance of a cotton plant to stress, modulating is decreasing and said second nucleic acid sequence encodes an RNA, which when transcribed 1. yields an RNA molecule capable of increasing the expression of a gene comprised in said cotton plant, said gene optionally being selected from PARP1, PARP2, FTA and FTB, e. g. by targeting genes involved in down-regulating the expression of these proteins, or 2. yields an RNA molecule capable of decreasing the expression of a gene comprised in said cotton plant, said gene being selected from NPT 1, PNC 1, Los5, NMA 1 and NMA2, for example by targeting this gene directly.

Also disclosed herein is the use of (a) the cotton plant or seed disclosed herein; (b) a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; b. a second nucleic acid sequence encoding an expression product of interest; and optionally c. a transcription termination and polyadenylation sequence; or (c) a nucleic acid sequence comprising at least 400 consecutive nucleotides of SEQ ID NO: 1 or SEQ ID NO: 2 or a nucleic acid sequence having at least 80% sequence identity thereto any of which confers stress inducibility on a transgene it is operably linked to; for expressing a transgene under stress conditions in cotton. The chimeric gene utilized in this use may be the chimeric gene as described herein above including all variations related thereto. Otherwise, all terms defining the present use have the meaning as described elsewhere in this application. For example, the term “stress” is defined as and includes all variations as described elsewhere.

Also disclosed herein is the use of (a) a nucleic acid sequence comprising at least 400 consecutive nucleotides of SEQ ID NO: 1 or SEQ ID NO: 2 or a nucleic acid sequence having at least 80% sequence identity thereto any of which confers stress inducibility on a transgene it is operably linked to; or (b) a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; b. a second nucleic acid sequence encoding an expression product of interest; and optionally c. a transcription termination and polyadenylation sequence for increasing a cotton plant's tolerance to stress or to detect a transgene in cotton fibers.

An increase of a cotton plant's tolerance to stress can be achieved by a method comprising operably linking the nucleic acid of (a) to a nucleic acid sequence encoding an expression product of interest which is involved in the response of a cotton plant to stress and introducing the resulting chimeric gene into a cotton plant. An increase of a cotton plant's tolerance is present e. g. if the cotton plant comprising the chimeric gene described herein survives longer or has an increased yield under stress conditions as described above compared to a cotton plant not comprising said chimeric gene.

Further disclosed herein are cotton fibers and cotton seed oil obtainable or obtained from the plants disclosed herein. Cotton fibers disclosed herein can be distinguished from other fibers by applying the detection method disclosed in WO2010/015423 and checking for the presence of the nucleic acid of (a) or chimeric gene of (b) in the fibers. Also disclosed herein are yarn and textiles made from the fibers disclosed herein as well as foodstuff and feed comprising or made of the cotton seed oil disclosed herein. A method to obtain cotton seed oil comprising harvesting cotton seeds from the cotton plant disclosed herein and extracting said oil from said seeds is also disclosed. Further, a method to produce cotton fibers comprising growing the cotton plant disclosed herein and harvesting cotton from said cotton plants is also disclosed.

Also disclosed herein is a method for protecting cotton fields from stress comprising (a) obtaining cotton plants comprising (i) a chimeric gene comprising

a. a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene; b. second nucleic acid sequence encoding an expression product of interest; and optionally c. a transcription termination and polyadenylation sequence; or (ii) the cotton plant cell described herein; or progeny thereof; and (b) planting said cotton plants in said field.

### THE FIGURES SHOW

FIG. 1: Pictures of the rd29::GUS transgenic cotton plants and wild type control under water stress conditions, relative soil water content (rswc) 10%.

FIG. 2: Expression profile of plants comprising rd29::GUS in comparison with wild-type plants. The expression level of the GUS gene from two independent rd29::GUS transgenic cotton plants designated 8901 and 6301 and wild type control was determined under water stress conditions. The GUS gene is not detected in the wild type control while the expression is detected in the two rd29::GUS cotton plants during water stress. The level of GUS expression returns to 0 after re-watering of the two rd29::GUS cotton plants.

FIG. 3: Expression analysis of leaf tissue of plants comprising the rd29a::PNC1 construct before and after application of drought stress.

### SEQUENCE LISTING

SEQ ID NO: 1: RD29 promoter with one deletion

SEQ ID NO: 2: RD29 promoter

SEQ ID NO: 3: primer naste 8

SEQ ID NO: 4: primer naste9

SEQ ID NO: 5: vector pGEM-T

SEQ ID NO: 6: vector pNVS10

SEQ ID NO: 7: vector pNVS11

SEQ ID NO: 8: Primer pNVS11FW

SEQ ID NO: 9: Primer pNVS11RV

SEQ ID NO: 10: vector pNVS122

SEQ ID NO: 11: Primer naste 79

SEQ ID NO: 12: vector pNVS123

SEQ ID NO: 13: Primer naste75

SEQ ID NO: 14: Primer naste 80

SEQ ID NO: 15: vector pNVS124

SEQ ID NO: 16: vector pTIBE10 comprising RD29 promoter with one deletion

SEQ ID NO: 17: vector pTIBE28 comprising RD29 promoter

SEQ ID NO: 18: GUS reporter gene with intron

SEQ ID NO: 19: nucleic acid sequence of the 3′ CaMV 35S terminator

SEQ ID NO: 20: nucleic acid sequence encoding the 2mepsps selectable marker cassette

SEQ ID NO: 21: nucleic acid sequence of the GUS gene with intron comprising the CaMV 3′35S terminator

SEQ ID NO: 22: nucleic acid sequence encoding the PNC1 protein

SEQ ID NO: 23: nucleic acid sequence encoding the NMA1 protein

SEQ ID NO: 24: nucleic acid encoding a microRNA against PARP1

SEQ ID NO: 25: nucleic acid encoding a hairpin RNA against PARP2

SEQ ID NO: 26: nucleic acid encoding a hairpinRNA directed against farnesytransferase α (FTA)

SEQ ID NO: 27: nucleic acid encoding a hairpinRNA directed against farnesytransferase β (FTB)

SEQ ID NO: 28: nucleic acid sequence encoding the Los5 protein

The following examples illustrate the invention. It is to be understood that the examples do not limit the spirit and scope of the subject-matter disclosed herein.

### EXAMPLES

Materials

Unless indicated otherwise, chemicals and reagents in the examples were obtained from Sigma Chemical Company, restriction endonucleases were from Fermentas or Roche-Boehringer, and other modifying enzymes or kits regarding biochemicals and molecular biological assays were from Qiagen, Invitrogen and Q-BIOgene. Bacterial strains were from invitrogen. The cloning steps carried out, such as, for example, restriction cleavages, agarose gel electrophores is, purification of DNA fragments, linking DNA fragments, transformation of E. coli cells, growing bacteria, multiplying phages and sequence analysis o f recombinant DNA, are carried out as described by Sambrook (1989). The sequencing of recombinant DNA molecules is carried out using ABI laser fluorescence DNA sequencer following the method of Sanger.

### Example 1

### Generation of Expression Constructs with a 933 bp Region from the rd29 Promoter (Comprising One Deletion) and a 934 bp Region from the rd29 Promoter, Respectively, Operably Linked to the GUS Reporter Gene

The 934 bp promoter region of the rd29a gene of A. thaliana, amplified from genomic Arabidopsis DNA using primer naste 8 5′ GCCCGGGCCATAGATGCAATTCAATCAAAC (SEQ ID NO: 3) and naste 9 5′GCGCTAGCCTCGAGTTAATTAAGATTTTTTTCTTTCCAATA (SEQ ID NO: 4) was cloned into the pGEM-T vector (SEQ ID NO: 5) resulting in the plasmid pNVS10 (SEQ ID NO: 6).

This plasmid contains 1 deletion in the rd29a promoter region compared to Yamaguchi-Shinozaki and Shinozaki (1994): one A missing at bp 3748.

At the 3′ end of the promoter region (i. e. in the 5′UTR) TCTTTGGAAA (SEQ ID NO:29) was changed into TCTTAATTAA (SEQ ID NO:30) to create a Pacl site for cloning reasons.

The CaMV 35S enhancer was added 3′ to the promoter region in pNVS10, resulting in pNVS11 (SEQ ID NO: 7).

For facilitating cloning of GOI with Ncol/Nhel, an Ncol site at the 5′ end of the rd29 promoter was eliminated, and a new Ncol site introduced at the 3′end of the rd29 promoter. This was done using primer pNVS11FW (5′CCTCATGACCATAGATGCAATTCAATCAAAC) (SEQ ID NO: 8) containing a BspHl site and pNVS11 RV (5′CCGCTAGCGCATCCATGGTCCAAAGATTTTTTTCTTTCAATAG) (SEQ ID NO: 9) containing an Ncol and Nhel site and pNVS11 as a PCR template. pNVS11 was digested with Ncol, Nhel, which cuts out the rd29a promoter region. The rd29 PCR product was cut with BspHl (compatible with Ncol) and Nhel. Ligation of the BspHl site into the Ncol site of the vector deleted the original Ncol site. Due to the sequence of the pNVS11Rv primer, an Ncol and an Nhel site were introduced behind the rd29a promoter, in front of the 3′ CaMV 35S. The resulting plasmid is pNVS122 (SEQ ID NO: 10).

To check if the deletion detected in the rd29 promoter resulted from sequencing errors in the TAIR database (The Arabidopsis Information Resource; http://www.arabidopsis.org/) sequence, or if it was due to a mistake in the original PCR fragment that was introduced to make pNVS10, 2 independent PCR reactions on genomic CTAB (cetrimonium bromide) DNA were performed, using the proofreading polymerase Phusion® with primers naste8 and naste9. Sequencing of PCR products showed that they did not have the deletion. To remove the deletion from pNVS122, a new primer naste 79 (5′CCGCTAGCGCATCCATGGTCCAAAGATTTTTTTCTTTCCAATAGAAGT) (SEQ ID NO: 11) was used in a PCR reaction, to replace primer pNVS11 RV. Using Phusion® polymerase, a PCR product was created with primers pNVS11FW and naste79 using genomic CTAB DNA as template. Both pNVS122 and the PCR product were cut with Spel and Ncol, and the correct PCR fragment was introduced into pNVS122 resulting in plasmid pNVS123 (SEQ ID NO: 12).

To facilitate further cloning in a T-DNA vector, an MCS-linker (Multiple Cloning Site) was introduced 5′ of the rd29a promoter. Primers naste75 5′-CATGCCCGGGCGCGCCTGTACAGCGGCCGCGAATTCGTTAACTCTAGAG CGATCGC-3′ (SEQ ID NO: 13) and naste80 5′-CCGGGCGATCGCTCTAGAGTTAACGAATTCGCGGCCGCTGTACAGGCG CGCCCGGG-3′ (SEQ ID NO: 14) were annealed, creating a linker with sticky ends. A 3-point-ligation was performed between a Pstl-Ncol fragment of pNVS11 +the sticky-end linker +an Eagl-Pstl fragment of pNVS123. The sticky end at the 5′ end of the linker is a CATG(C) overhang, which anneals to the Ncol site, but this ligation abolishes the Ncol site in the resulting plasmid pNVS124 (SEQ ID NO: 15).

Generation of the Expression Vectors:

The rd29a promoter comprising one deletion was amplified from pNVS11 and was cloned for one step cloning in an intermediate vector.

The rd29 promoter fragment with one deletion (SEQ ID NO: 1), the GUS gene with intron (SEQ ID NO: 18) and the 3′ CaMV 35S terminator (SEQ ID NO: 19) were assembled in a backbone vector which contains the 2mepsps selectable marker cassette (SEQ ID NO: 20) to result in expression vector pTIBE10 (SEQ ID NO: 16).

Expression vector pTIBE28 (SEQ ID NO: 17) contains the Spel-Ncol rd29a promoter without deletion (comprising SEQ ID NO: 2) linked to the Nhel-Ncol GUS gene with intron and the CaMV 3′35S terminator (SEQ ID NO: 21). Both fragments, i. e. GUS-3′35S terminator and Spel-Ncol rd29a promoter were assembled in a a vector which contains the 2mepsps selectable marker resulting in pTIBE28 (SEQ ID NO: 17).

### Example 2

### Generation of Transgenic Plants Comprising rd29-GUS

In a next step the recombinant vector comprising the expression cassettes of example 1, i. e. vectors pTIBE10 and pTIBE28, were used to stably transform Gossypium hirsutum coker 312 using the embryogenic callus transformation protocol.

Control plants are null segregants of the Gossypium hirsutum coker 312 rd29-GUS transgenic lines.

### Example 3

### Drought Stress Inducibility of rd29::GUS

β-glucuronidase activity of plants transformed with pTIBE28 was monitored in planta with the chromogen ic substrate X-Gluc (5-bromo-4-Chloro-3-indolyl-β-D-glucuronic acid) during corresponding activity assays (Jefferson R A et al (1987) EMBO J. 20;6(13):3901-7). For determination of promoter activity plant tissue is dissected, embedded, stained and analyzed as described (e.g., Pien S. et al (2001) PNAS 98(20):11812-7), Thus, the activity of beta-glucuronidase in the transformed plants was witnessed by the presence of the blue color due to the enzymatic metabolism of the substrate X-Gluc.

After growing the plants for about 30 days with sufficient water supply plants were subjected to drought stress by not watering them any more.

Expression of the GUS reporter gene was monitored over 5 days, re-watering taking place on day 5.

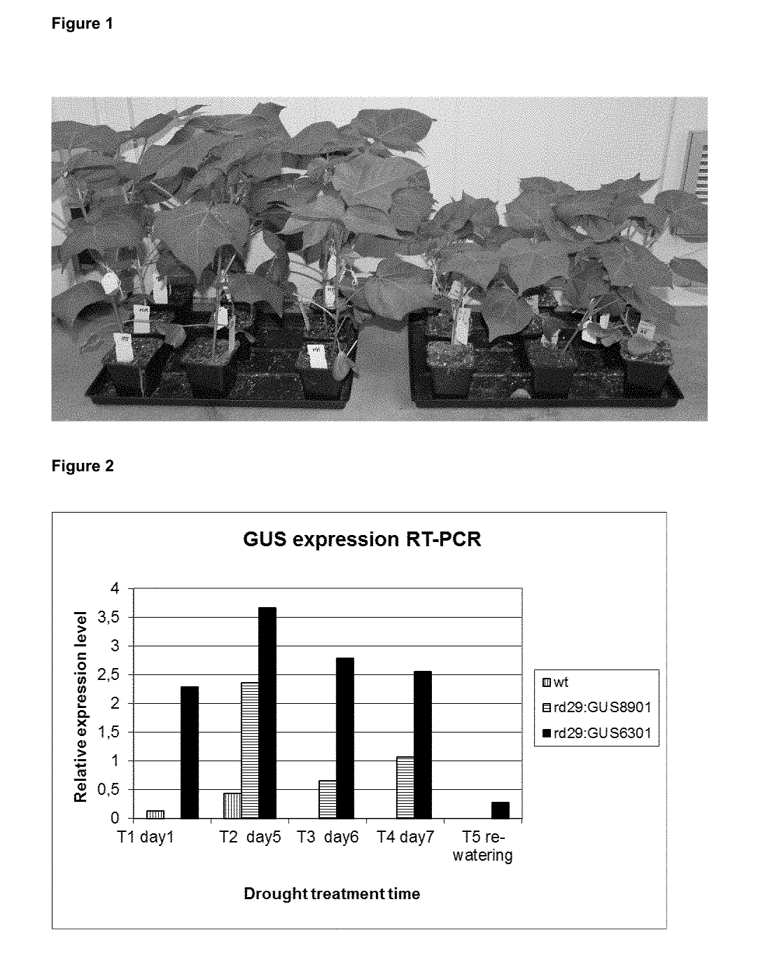

After five days of drought stress, stressed plants were significantly smaller than non-stressed plants (see FIG. 1).

FIG. 2 shows the expression profile of two plant lines comprising rd29::GUS from pTIBE28 in comparison with wild-type plants.

As apparent from the figure, both transgenic lines expressed the GUS reporter gene under control of the rd29 promoter under drought stress conditions. Expression is abolished upon re-watering the plants. Thus, the rd29 promoter may be used for expression of genes under stress conditions.

### Example 4

### Expression of Chimeric Genes Comprising rd29 do not Impair Fertility and Plant Vigor

The PNC1 (SEQ ID NO: 22), NMA1 (SEQ ID NO: 23) and Los5 (SEQ ID NO: 28) genes as well as a nucleic acid encoding a micro RNA directed against the PARP1 gene (miPARP1) (SEQ ID NO: 24) and a hairpin construct against the PARP2 gene (SEQ ID NO: 25) were placed under control of the rd29a promoter. The resulting constructs were individually transformed into cotton and plants regenerated. Cotton T0 plants containing the chimeric genes were fertile and produced viable T1 seeds. A germination test with the T1 seeds gave between 90 and 100% germination (20 seeds were sown and scored for germination, all T1 plants had a normal vigor). From the T1 plants no fertility issue was observed, the homozygous and azygous plants produced more than 400 T2 seeds per plants. A germination test with the T2 seeds gave between 80 and 100% germination (15 to 20 seeds were sown and scored for germination, all T2 plants had a normal vigor)

a

| | | | | Number of | |
|---|---|---|---|---|---|
| | | | | seeds | |
| | Event | Gener- | Geno- | generated | % |
| Construct | T0 | ation | type | per plants | germination |
| Rd29a:PNC1 | 04801 | T1 | Hh | 162 | 100 |
| Rd29a:PNC1 | 04801 | T2 | HH | 938 | 90 |
| Rd29a:PNC1 | 04801 | T2 | hh | 868 | 100 |
| Rd29a:PNC1 | 02501 | T1 | Hh | 166 | 100 |
| Rd29a:PNC1 | 02501 | T2 | HH | 524 | 90 |
| Rd29a:PNC1 | 02501 | T2 | hh | 529 | 100 |
| rd29a:miRPARP1 | 10701 | T1 | Hh | 156 | 100 |
| rd29a:miRPARP1 | 10701 | T2 | HH | 582 | 90 |
| rd29a:miRPARP1 | 10701 | T2 | hh | 544 | 100 |
| rd29a:miRPARP1 | 01901 | T1 | Hh | 203 | 95 |
| rd29a:miRPARP1 | 01901 | T2 | HH | 567 | 100 |
| rd29a:miRPARP1 | 01901 | T2 | hh | 536 | 80 |
| rd29aNMA1 | 02102 | T1 | Hh | 653 | 100 |
| rd29aNMA1 | 02102 | T2 | HH | 616 | 100 |
| rd29aNMA1 | 02102 | T2 | hh | 586 | 93 |
| rd29aNMA1 | 02204 | T1 | Hh | 619 | 100 |
| rd29aNMA1 | 02204 | T2 | HH | 499 | 100 |
| rd29aNMA1 | 02204 | T2 | hh | 512 | 100 |

b

| | | | | Number of | |
|---|---|---|---|---|---|
| | | | | seeds | |
| | | Gener- | Geno- | generated | % |
| Construct | Event | ation | type | per plants | Germination |
| Rd29a:LOS5 | 00801 | T0 | Hh | 352 | 95 |
| Rd29a:LOS5 | 006901 | T0 | Hh | 337 | 95 |
| Rd29a:LOS5 | 10901 | T0 | Hh | 327 | 95 |
| Rd29a:hpPARP2 | 01901 | T0 | Hh | 310 | 90 |
| Rd29a:hpPARP2 | 02203 | T0 | Hh | 375 | 100 |
| Rd29a:hpPARP2 | 06202 | T0 | Hh | 311 | 95 |

Table 1a and b: fertility of T1 and T2 cotton plants comprising a chimeric gene according to the invention,
a: six plants per construct examined;
b: three plants per construct examined

### Example 5

### Drought Stress Inducibility of a rd29::PNC1 Chimeric Gene

Plants comprising a chimeric gene comprising the PNC1 coding sequence were made as described above. Initially, the plants were watered 2 times per week. Drought stress (no watering for five days) was applied to three three week old plants (pncl 1-1, pncl 1-2, and pncl 1-3). The PNC1 expression level was quantified before and after drought stress of the three rd29a::PNC1 plants transformed with the chimeric gene.

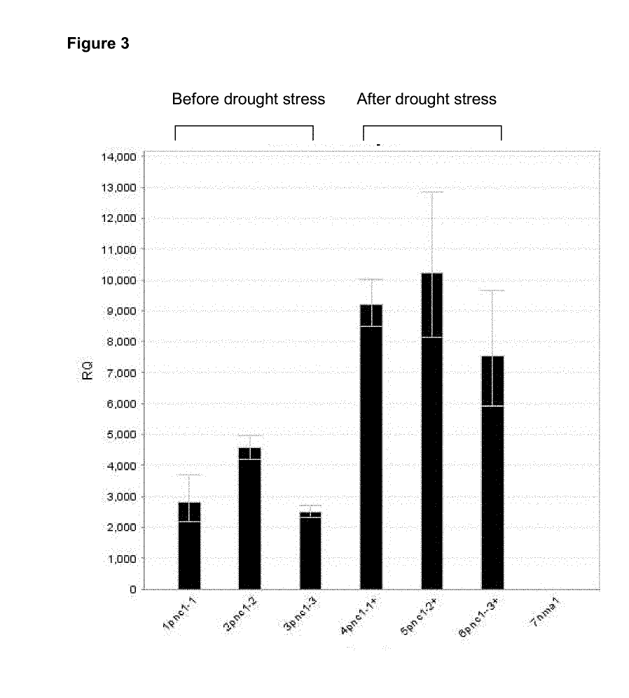

Leaf tissue was harvested from each rd29a::PNC1 plant before and after drought stress and assayed for PNC1 expression level using the quantitative PCR (qPCR) (see FIG. 3). Three normally watered plants comprising the chimeric gene were used as base l ine to calculate variation. After drought stress the transgenic plant showed a 2 to 3 fold increase of the PNC1 expression level compared to the non-stressed transgenic plants.

Plants comprising the other chimeric genes as indicated in table 1 are examined for stress inducibility as described above. It is shown that expression of the genes is induced after application of drought stress.

### Example 6

### Expression of Further Chimeric Genes According to the Invention in Cotton

Plants comprising a chimeric gene comprising a nucleic acid sequence encoding a hairpin construct directed against farnesyltransferase a (SEQ ID NO: 26) and farnesyltransferase β (SEQ ID NO: 27) were made and grown in the greenhouse.

The plants are shown to be fertile. Plants are examined for stress inducibility as described above. It is shown that expression of the genes is induced after application of drought stress.

### REFERENCES

Ackerson, R. C. (1982). Synthesis and Movement of Abscisic Acid in Water-Stressed Cotton Leaves. Plant Physiol 69, p. 609-613.

Ahmad et al. (2010). Simultaneous expression of choline oxidase, superoxide dismutase and ascorbate peroxidase in potato plant chloroplasts provides synergistically enhanced protection against various abiotic stresses. Physiol. Plant. 138 (4), p 520-533.

Allen, R. D. (2010). Opportunities for Engineering Abiotic Stress Tolerance in Cotton Plants; in Biotechnology in Agriculture and Forestry, Springer Verlag, Berlin, p: 127-160.

Behnam et al. (2007). Arabidopsis rd29A::DREB1A enhances freezing tolerance in transgenic potato. Plant Cell Rep, p. 1275-1282.

Braasch and Corey (2001). Locked nucleic acid (LNA): fine-tuning the recognition of DNA and RNA. Chem Biol 8(1), p. 1-7.

Carrington & Freed 1990. Cap-independent enhancement of translation by a plant potyvirus 5′ nontranslated region. J. Virol. 64, p. 1590-1597.

Chaudhary et al. (2008). EVOLUTION & DEVELOPMENT 10(5), p. 567-582.

Hong et al. (2006). Sci China C Life Sci, p: 436-45.

Liu, H. and Stone, S. L. (2010). Abscisic Acid Increases Arabidopsis ABI5 Transcription Factor Levels by Promoting KEG E3 Ligase Self-Ubiquitination and Proteasomal Degradation. Plant Cell 22, p. 2630-2641.

Logan, Edwards and Saunders (Editors; Real-Time PCR: Current Technology and Applications, Caister Academic Press 2009, ISBN: 978-1-904455-39-4.

López-Carbonell and Jáuregui (2005). A rapid method for analysis of abscisic acid (ABA) in crude extracts of water stressed Arabidopsis thaliana plants by liquid chromatography-mass spectrometry in tandem mode. Plant Physiol Biochem 43, p. 407-411.

Mansfield, T. A. (1987) Hormones as regulators of water balance. In Plant hormones and their role in growth and development, R. D. Davies, ed (Dordrecht, The Netherlands: Martinus Nijhoff Publishers), p. 411-430.

Masood et al. (2006). Differential response of antioxidant enzymes to salinity stress in two varieties of Azolla (Azolla pinnate and Azolla tilieuloides) . Env. Exp. Bot. 58, p. 216-222.

Mittler (2002). Oxidative stress, antioxidants and stress tolerance. Trends Plant Sci. 7, p, 405-410.

Needleman and Wunsch (1970). A general method applicable to the search for similarities in the amino acid sequence of two proteins. J. Mol. Biol., 48, p.443-453.

Pearson and Lipman (1988). Improved tools for biological sequence comparison. Proc. Natl. Acad. Sci 85, p.2444-48.

Pellegrineschi et al. (2004). Stress-induced expression in wheat of the Arabidopsis thaliana DREB1A gene delays water stress symptoms under greenhouse conditions. Genome 47, p. 493-500.

Sambrook, J. F., Russell, D. W. and Irwin, N. (2000). Molecular Cloning: A Laboratory Manual, 3rd edition Volumes 1 , 2, and 3. Cold Spring Harbor Laboratory Press.

Seo M. and Koshiba, T. (2002). Complex regulation of ABA biosynthesis in plants. Trends Plant Sci.7 (1): 41-48.

Wang et al. (2005). Molecular tailoring of farnesylation for plant drought tolerance and yield protection. The Plant Journal 43, p. 413-24.

Waterman, M. S. (1995). Introduction to Computational Biology: Maps, sequences and genomes. Chapman & Hall. London.

Xiong et al. (2001). The Arabidopsis LOS5/ABA3 Locus Encodes a Molybdenum Cofactor Sulfurase and Modulates Cold Stress- and Osmotic Stress-Responsive Gene Expression. The Plant Cell 13, p. 2063-2083.

Yamaguchi-Shinozaki, K. and Shinozaki, K. (1993). Characterization of the expression of a desiccation-responsive rd29 gene of Arabidopsis thaliana and alanysis of its promoter in transgenic plants. Mol Gen Genet 236, p. 331-340.

Yamaguchi-Shinozaki and Shinozaki (1994). A Novel cis-Acting Element in an Arabisopsis Gene Is Involved in Responsiveness to Drought, Low-Temperature, or High-Salt Stress. The Plant Cell 6, p. 251-264.

Zanoni et al. (2009) Nature 460, p:264-269; see also Nature Protocols: mRNA expression analysis by Real-Time PCR; ISSN: 1754-2189.

1. (canceled)

2. A cotton plant cell comprising a chimeric gene comprising
(a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene;
(b) a second nucleic acid sequence encoding an expression product of interest; and optionally
(c) a transcription termination and polyadenylation sequence.

3. The cotton plant cell of claim 2, wherein said expression product of interest is
(a) a protein or peptide which is involved in the response of a cotton plant to stress or
(b) an RNA molecule capable of modulating the expression of a gene comprised in said cotton plant, wherein said gene comprised in said cotton plant is involved in the response of said cotton plant to stress.

4. The cotton plant cell of claim 3, wherein said protein optionally involved in the response of a cotton plant to stress is selected from NPT 1, PNC 1, NMA 1, NMA2, Los5 and proteins involved in oxidative stress such as selected from choline oxidase, superoxide dismutase and ascorbate peroxidase.

5. The cotton plant cell of claim 3, wherein said gene optionally involved in the response of a cotton plant to stress is selected from NPT 1, PNC 1, NMA 1, NMA2, PARP1, PARP2, Los5, FTA, FTB and genes involved in oxidative stress selected from choline oxidase, superoxide dismutase and ascorbate peroxidase.

6. The cotton plant cell of claim 3, wherein modulating is increasing and said second nucleic acid sequence encodes an RNA, which when transcribed
(a) yields an RNA molecule capable of increasing the expression of a gene comprised in said cotton plant, said gene being selected from NPT 1, PNC 1, Los5, NMA 1 and NMA2 or
(b) yields an RNA molecule capable of decreasing the expression of a gene comprised in said cotton plant, said gene being selected from PARP1, PARP2, FTA and FTB.

7. The the cotton plant cell of claim 3, wherein modulating is decreasing and said second nucleic acid sequence encodes an RNA, which when transcribed
(a) yields an RNA molecule capable of increasing the expression of a gene comprised in said cotton plant, said gene being selected from PARP1, PARP2, FTA and FTB or
(b) yields an RNA molecule capable of decreasing the expression of a gene comprised in said cotton plant, said gene being selected from NPT 1, PNC 1, Los5, NMA 1 and NMA2.

8. The cotton plant cell of claim 3, wherein said RNA molecule comprises a first and second RNA region wherein
(a) said first RNA region comprises a nucleotide sequence of at least 19 consecutive nucleotides having at least about 94% sequence identity to the nucleotide sequence of said gene comprised in said cotton plant;
(b) said second RNA region comprises a nucleotide sequence complementary to said 19 consecutive nucleotides of said first RNA region; and
(c) said first and second RNA region are capable of base-pairing to form a double stranded RNA molecule between at least said 19 consecutive nucleotides of said first and second region.

9. The cotton plant cell of claim 2, wherein said first nucleic acid sequence comprises the nucleotide sequence of SEQ ID NO: 1 or SEQ ID NO: 2.

10. The cotton plant cell of claim 2, wherein said first nucleic acid sequence consists of SEQ ID NO: 1 or SEQ ID NO: 2.

11. The cotton plant cell of claim 2, wherein said stress is water stress, cold stress, high-salt stress or the application of ABA.

12. The cotton plant cell of claim 11, wherein said water stress is drought stress.

13. A cotton plant or seed thereof or cotton plant part comprising
a chimeric gene comprising
(a) a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene;
(b) a second nucleic acid sequence encoding an expression product of interest; and optionally
(c) a transcription termination and polyadenylation sequence.

14. A cotton fiber obtainable from the cotton plant or seed thereof of claim 13.

15. A method of expressing a transgene in cotton under stress conditions comprising:
(a) introducing or introgressing a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene, a second nucleic acid sequence encoding an expression product of interest, and optionally a transcription termination and polyadenylation sequence into a cotton plant and growing the plant.
(b) having said plant exposed to stress.

16. A method of producing a cotton plant comprising:
(a) introducing or introgressing a chimeric gene comprising a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene, a second nucleic acid sequence encoding an expression product of interest, and optionally a transcription termination and polyadenylation sequence; OR
(b) growing the plant of claim 13 or growing a plant from the seed of claim 13.

17. A method of detecting the expression of a transgene under stress conditions comprising
(a) providing the cotton plant cell of claim 2 or the plant of claim 13, wherein said expression product of interest is the transgene;
(b) having the plant exposed to stress;
(c) detecting the expression of the transgene.

18. A method for modulating the resistance of a cotton plant to stress comprising
(a) introducing or introgressing into a cotton plant a chimeric gene comprising
i. a first nucleic acid sequence comprising at least 400 consecutive nucleotides of SEQ ID NO: 1 or SEQ ID NO: 2 or a nucleic acid sequence having at least 80% sequence identity thereto any of which confers stress inducibility on said chimeric gene;
ii. a second nucleic acid sequence encoding an expression product of interest which is optionally involved in the response of a cotton plant to stress; and optionally
iii. a transcription termination and polyadenylation sequence;
(b) having said chimeric gene expressed under stress conditions;
wherein the resistance of the stress of the cotton plant comprising the chimeric gene is modulated in comparison with the resistance to stress of a wild-type cotton plant.

19. (canceled)

20. A method of expressing a transgene in cotton under stress conditions comprising:
(a) growing the cotton plant of claim 13 or growing a plant from the seed of claim 13;
(b) having said plant exposed to stress.
